(19) United States
(12) Patent Application Publication
(10) Pub. No.: US 2022/0392602 A1
(43) Pub. Date: Dec. 8, 2022

(54) SYSTEM AND METHOD FOR USER-CONFIGURABLE PRESSURE LIMITING BEHAVIOR FOR FLUID INJECTION DEVICES

(71) Applicant: BAYER HEALTHCARE LLC, WHIPPANY, NJ (US)

(72) Inventors: MICHAEL MCDERMOTT, BERLIN (DE); COREY KEMPER, PITTSBURGH, PA (US)

(21) Appl. No.: 17/775,970

(22) PCT Filed: Nov. 18, 2020
(86) PCT No.: PCT/US2020/060956

§ 371 Date: May 11, 2022

Related U.S. Application Data
(60) Provisional application No. 62/938,436, filed on Nov. 21, 2019.

(51) Int. Cl. G16H 20/17

(52) U.S. Cl. CPC G16H 20/17

(57) ABSTRACT

A system and method are disclosed for user- or system-configurable and adaptive pressure limiting behavior in fluid injection systems/devices. A fluid injector system may include at least one fluid injector device, at least one user interface, and a control device comprising at least one processor. The at least on processor may be programmed or configured to: receive a maximum pressure limit for an injection procedure, receive a programmed fluid flow rate for the injection procedure, receive a maximum fluid flow rate reduction input for the injection procedure, wherein the maximum fluid flow rate reduction input is selected by a user via the at least one user interface, and receive a pressure limit sensitivity input for the injection procedure, wherein the pressure limit sensitivity input is selected by the user via the at least one user interface. The at least one processor may also be configured to control the at least one fluid injector device to perform the injection procedure based on the maximum pressure limit, the programmed fluid flow rate, the maximum fluid flow rate reduction input, and the pressure limit sensitivity input.

### CROSS-REFERENCE TO RELATED APPLICATIONS

This application claims the benefit of U.S. Provisional Patent Application No. 62/938,436 filed on 21 Nov. 2019, the disclosure of which is hereby incorporated by reference in its entirety.

### BACKGROUND OF THE DISCLOSURE

### 1. Field of the Disclosure

This disclosure relates generally to systems, devices, products, apparatus, and methods that are used for configurable/customizable pressure limiting behavior for fluid injection devices. Configuration and/or customization of the pressure limiting behavior may be performed by a user or by the system based on a series of inputted parameters.

### 2. Description of the Related Art

In many medical diagnostic and therapeutic procedures, a medical practitioner, such as a physician or radiologist, injects a patient with one or more fluids using a powered fluid injector system. In recent years, a number of powered fluid injector systems for pressurized injection of fluids have been developed for use in procedures such as angiography, computed tomography (CT), molecular imaging (such as PET imaging), and magnetic resonance imaging (MRI).

Patient catheters used in any powered injection procedure include pressure ratings which are selected to prevent intravenous (IV) site failure during the injection procedure. Depending on factors such as, e.g., the type of procedure, the age of the patient, etc., fluid injector systems have pre-programmed pressure limits to ensure that the fluid pressure does not exceed the rating of the patient catheter used at the injection site. For some catheters commonly used at sensitive injection sites such as, e.g., Central Venous Catheters, Peripherally Inserted Central Catheter (PICC), etc., there is a desire to ensure that the fluid pressure does not exceed the pressure limit. However, even with the pre-programmed pressure limits, the maximum recorded pressure may still be in excess of these pre-programmed pressure limits in some cases.

Alternatively, in other procedures where slight overpressure may not be as undesirable, it may be more advantageous to maintain the fluid flow rate as close as possible to the commanded injection flow rate in order to ensure that the injection procedure is successfully completed. However, in current fluid injector systems, when the pre-programmed pressure limit is breached, the fluid flow is significantly reduced so as to quickly lower the pressure in the system. Unfortunately, this abrupt reduction in fluid flow may negatively affect the injection procedure and may necessitate that the injection be aborted and/or repeated.

Despite these potential issues, the pressure limits are typically determined by the manufacturer and hard-coded into the system, and the medical practitioner is unable to configure or customize the pressure limiting behavior to suit a particular injection procedure. Accordingly, there is a need in the art to improve the setting of pressure limiting behavior for fluid injection devices.

### SUMMARY OF THE DISCLOSURE

Accordingly, provided are systems, devices, products, apparatus, and/or methods for a fluid injector system that provides for improved pressure limiting behaviors.

In some non-limiting embodiments or aspects, a fluid injector system may be configured for administering at least one fluid. The fluid injector system may include: at least one fluid injector device; at least one user interface; and a control device in communication with the at least one user interface, the control device utilize at least one of internal inputs and external inputs to instruct the at least one fluid injector device to perform a fluid injection procedure according to a profile that determines prioritization of maintaining flow rate or limiting a fluid delivery pressure. The at least one of internal inputs and external inputs may be selected from the group consisting of receiving a maximum pressure limit for a fluid injection procedure; receiving a programmed fluid flow rate for the fluid injection procedure; receiving a maximum fluid flow rate reduction input for the fluid injection procedure, wherein the maximum fluid flow rate reduction input is selected by a user via the at least one user interface or by the system based off of at least one of patient information and system injection parameters; receiving a pressure limit sensitivity input for the fluid injection procedure, wherein the pressure limit sensitivity input is selected by the user via the at least one user interface; and or any combination thereof, and wherein the control device is further configured to instruct the at least one fluid injector device to perform the injection procedure based on one or more of the maximum pressure limit, the programmed fluid flow rate, the maximum fluid flow rate reduction input, and the pressure limit sensitivity input.

In some non-limiting embodiments or aspects, the at least one user interface may include at least one graphical user interface and wherein the at least one graphical user interface displays a flow rate profile of the fluid injection procedure, wherein the displayed flow rate profile further indicates one or more of the maximum pressure limit, the programmed fluid flow rate, the maximum fluid flow rate reduction input, and the pressure limit sensitivity input.

In some non-limiting embodiments or aspects, the at least one processor may be further programmed or configured to reduce the fluid flow rate of the at least one fluid injector device to provide a reduction in flow rate below the programmed fluid flow rate when a predetermined threshold pressure relative to the maximum pressure limit is reached.

In some non-limiting embodiments and aspects, the reduction in fluid flow rate may have one of a linear or non-linear flow reduction profile.

In some non-limiting embodiments and aspects, the reduction of fluid flow rate is based on at least one input provided by a user of the fluid injector device. For example, in certain embodiments the at least one input provided by the user is provided for all fluid injection procedures performed by the fluid injector system, provided for each fluid injection procedure, or provided for a limited set of fluid injection procedures. In various embodiments, the at least one user input provided by the user is selected by the user from a series of inputs saved in a protocol library or inputted manually by the user prior to initiation of the fluid injection procedure.

In some non-limiting embodiments and aspects, the reduction in fluid flow rate is based on at least one internally sourced or externally sourced input. For example, in certain embodiments the at least one internally sourced or externally sourced input is selected from the group consisting of historical injector data, historical patient data, information from one or more barcodes, one or more lookup tables, and combinations of any thereof. The one or more barcodes is located on a patient record, the fluid injector, a medical fluid container, a sterile disposable, and combinations thereof.

In some non-limiting embodiments or aspects, the maximum fluid flow rate reduction input may be user- or system-selectable within a range of 0% to 100% of the programmed fluid flow rate. In some non-limiting embodiments or aspects, the at least one processor may be programmed or configured to abort the injection procedure if the maximum fluid flow rate reduction input of a critical number (e.g., 0%) is selected and a pressure of the at least one fluid is detected to equal or exceed the maximum pressure limit. In other embodiments, the fluid injection may be aborted when a predetermined threshold is reached or exceeded, where the threshold may be a percentage of the programmed pressure limit.

In some non-limiting embodiments or aspects, the pressure limit sensitivity input may be user- or system-selectable within a configurable range from low sensitivity to high sensitivity.

In some non-limiting embodiments or aspects, the at least one processor may be programmed or configured to abort the injection procedure if specific configuration criteria are met and the pressure is detected to equal or exceed the predetermined pressure threshold.

In some non-limiting embodiments or aspects, a method of user- or system-configurable pressure limiting behavior for a fluid injector system may be configured for administering at least one fluid. The method may comprise providing at least one of internal inputs and external inputs to a fluid injector system; prioritizing maintaining flow rate or limiting a fluid delivery pressure based on the at least one of the internal inputs and the external inputs; developing an injection profile based on the prioritizing; and instructing an at least one fluid injector device to perform a fluid injection procedure according to the injection profile. According to certain non-limiting embodiments or aspects, wherein providing the at least one of internal inputs and external inputs comprises an operation selected from the group consisting of inputting or selecting a maximum pressure limit for an injection procedure into at least one user interface in communication with a control device of the fluid injector system, wherein the control device comprises at least one processor; inputting or selecting a programmed fluid flow rate for injecting at least one fluid from the at least one fluid injector device; inputting or selecting a maximum fluid flow rate reduction input for the injection procedure based off of at least one of patient information and system injection parameters; inputting or selecting a pressure limit sensitivity input for the injection procedure; and combinations of any thereof. According to certain non-limiting embodiments or aspects, the method may further comprise generating, by the control device, instructions to the fluid injector system to perform the injection procedure based on the maximum pressure limit, the programmed fluid flow rate, the maximum fluid flow rate reduction input, and the pressure limit sensitivity input.

In some non-limiting embodiments or aspects, the at least on user interface may include at least one graphical user interface and wherein the at least one graphical user interface displays a flow rate profile of the fluid injection procedure, wherein the displayed flow rate profile further indicates one or more of the maximum pressure limit, the programmed fluid flow rate, the maximum fluid flow rate reduction input, and the pressure limit sensitivity input.

In some non-limiting embodiments or aspects, the method may further include reducing, by the control device, the fluid flow rate of the at least one fluid injector device to provide a reduction in flow rate below the programmed fluid flow rate when a predetermined threshold pressure relative to the maximum pressure limit is reached.

In some non-limiting embodiments or aspects, reducing the fluid flow rate is based on at least one input provided by a user of the fluid injector device. For example, in certain embodiments, the at least one input provided by the user is provided for all fluid injection procedures performed by the fluid injector system, provided for each fluid injection procedure, or provided for a limited set of fluid injection procedures. In certain embodiments, the at least one user input provided by the user is selected by the user from a series of inputs saved in a protocol library or inputted manually by the user prior to initiation of the fluid injection procedure.

In some non-limiting embodiments or aspects, reducing the fluid flow rate is based on at least one internally sourced or externally sourced input. For example, in certain embodiments, the at least one internally sourced or externally sourced input is selected from the group consisting of historical injector data, historical patient data, information from one or more barcodes, one or more lookup tables, and combinations of any thereof. The one or more barcodes is located on a patient record, the fluid injector, a medical fluid container, a sterile disposable, and combinations thereof.

In some non-limiting embodiments or aspects, inputting or selecting the maximum fluid flow rate reduction input may include inputting or selecting a user- or system-selectable input within a range of 0% to 100% of the programmed fluid flow rate.

In some non-limiting embodiments or aspects, the method may further comprise aborting the injection procedure if a maximum fluid flow rate reduction input of a critical number (e.g., 0%) is selected and a pressure of the at least one fluid is detected to exceed the maximum pressure limit. In other embodiments, the fluid injection may be aborted when a predetermined threshold is reached or exceeded, where the threshold may be a percentage of the programmed pressure limit.

In some non-limiting embodiments or aspects, inputting or selecting the pressure limit sensitivity input may include inputting or selecting a user- or system-selectable input within a configurable range from low sensitivity to high sensitivity.

In some non-limiting embodiments or aspects, the method may further comprise aborting the injection procedure if specific configuration criteria are met and the pressure is detected to exceed the predetermined pressure threshold.

Further non-limiting embodiments are set forth in the following numbered clauses:

Clause 1. A fluid injector system configured for administering at least one fluid, the fluid injector system comprising: at least one fluid injector device; at least one user interface; and a control device in communication with the at least one user interface, the control device comprising at least one processor programmed or configured to utilize at least one of internal inputs and external inputs to instruct the at least one fluid injector device to perform a fluid injection procedure according to a profile that determines prioritization of maintaining flow rate or limiting a fluid delivery pressure.

Clause 2 The fluid injector system of clause 1, wherein the at least one of internal inputs and external inputs are selected from the group consisting of: receiving a maximum pressure limit for a fluid injection procedure; receiving a programmed fluid flow rate for the fluid injection procedure; receiving a maximum fluid flow rate reduction input for the fluid injection procedure, wherein the maximum fluid flow rate reduction input is selected by a user via the at least one user interface or by the system based off of at least one of patient information and system injection parameters; receiving a pressure limit sensitivity input for the fluid injection procedure, wherein the pressure limit sensitivity input is selected by the user via the at least one user interface; and any combination thereof, wherein the control device is further configured to instruct the at least one fluid injector device to perform the injection procedure based on one or more of the maximum pressure limit, the programmed fluid flow rate, the maximum fluid flow rate reduction input, and the pressure limit sensitivity input.

Clause 3. The fluid injector system of clause 1 or 2, wherein the at least one user interface comprises at least one graphical user interface and wherein the at least one graphical user interface displays a flow rate profile of the fluid injection procedure, wherein the displayed flow rate profile further indicates one or more of the maximum pressure limit, the programmed fluid flow rate, the maximum fluid flow rate reduction input, and the pressure limit sensitivity input.

Clause 4. The fluid injector system of any one of clauses 1 to 3, wherein the at least one processor is programmed or configured to limiting a fluid delivery pressure by reducing a flow rate of the fluid, diluting a more viscous fluid with a less viscous fluid, or combinations thereof.

Clause 5. The fluid injector system of any one of clauses 1 to 3, wherein the at least one processor is further programmed or configured to reduce the fluid flow rate of the at least one fluid injector device to provide a reduction in flow rate below the programmed fluid flow rate when a predetermined threshold pressure relative to the maximum pressure limit is reached.

Clause 6. The fluid injector system of clause 5, wherein the reduction in fluid flow rate has one of a linear or non-linear flow reduction profile.

Clause 7. The fluid injector system of clause 5, wherein the reduction of fluid flow rate is based on at least one input provided by a user of the fluid injector device.

Clause 8. The fluid injector system of clause 7, wherein the at least one input provided by the user is provided for all fluid injection procedures performed by the fluid injector system, provided for each fluid injection procedure, or provided for a limited set of fluid injection procedures.

Clause 9. The fluid injector system of clause 8, wherein the at least one user input provided by the user is selected by the user from a series of inputs saved in a protocol library or inputted manually by the user prior to initiation of the fluid injection procedure.

Clause 10. The fluid injector system of clause 5, wherein the reduction in fluid flow rate is based on at least one internally sourced or externally sourced input.

Clause 11. The fluid injector system of clause 10, wherein the at least one internally sourced or externally sourced input is selected from the group consisting of historical injector data, historical patient data, information from one or more barcodes, one or more lookup tables, and combinations of any thereof, wherein the one or more barcodes is located on a patient record, the fluid injector, a medical fluid container, a sterile disposable, and combinations thereof.

Clause 12. The fluid injector system of any one of clauses 2 to 11, wherein the maximum fluid flow rate reduction input is user- or system-selectable within a range of 0% to 100% of the programmed fluid flow rate.

Clause 13. The fluid injector system of any one of clauses 2 to 12, wherein the at least one processor is programmed or configured to abort the injection procedure if the maximum fluid flow rate reduction input of 0% is selected and a pressure of the at least one fluid is detected to equal or exceed the maximum pressure limit.

Clause 14. The fluid injector system of any one of clauses 2 to 13, wherein the pressure limit sensitivity input is user- or system-selectable within a configurable range from low sensitivity to high sensitivity.

Clause 15. The fluid injector system of any one of clauses 5 to 14, wherein the at least one processor is programmed or configured to abort the injection procedure if specific configuration criteria are met and the pressure is detected to equal or exceed the predetermined pressure threshold.

Clause 16. A method of user- or system-configurable pressure limiting behavior for a fluid injector system configured for administering at least one fluid comprising: providing at least one of internal inputs and external inputs to a fluid injector system; prioritizing maintaining flow rate or limiting a fluid delivery pressure based on the at least one of the internal inputs and the external inputs; developing an injection profile based on the prioritizing; and instructing an at least one fluid injector device to perform a fluid injection procedure according to the injection profile.

Clause 17. The method of clause 16, wherein providing the at least one of internal inputs and external inputs comprises an operation selected from the group consisting of: inputting or selecting a maximum pressure limit for an injection procedure into at least one user interface in communication with a control device of the fluid injector system, wherein the control device comprises at least one processor; inputting or selecting a programmed fluid flow rate for injecting at least one fluid from the at least one fluid injector device; inputting or selecting a maximum fluid flow rate reduction input for the injection procedure based off of at least one of patient information and system injection parameters; inputting or selecting a pressure limit sensitivity input for the injection procedure; and combinations of any thereof.

Clause 18. The method of clause 17 further comprising generating, by the control device, instructions to the fluid injector system to perform the injection procedure based on the maximum pressure limit, the programmed fluid flow rate, the maximum fluid flow rate reduction input, and the pressure limit sensitivity input.

Clause 19. The method of any one of clauses 16 to 18, wherein the at least on user interface comprises at least one graphical user interface and wherein the at least one graphical user interface displays a flow rate profile of the fluid injection procedure, wherein the displayed flow rate profile further indicates one or more of the maximum pressure limit, the programmed fluid flow rate, the maximum fluid flow rate reduction input, and the pressure limit sensitivity input.

Clause 20. The method of any one of clauses 16 to 19, further comprising limiting a fluid delivery pressure by reducing a flow rate of the fluid, diluting a more viscous fluid with a less viscous fluid, or combinations thereof.

Clause 21. The method of any one of claims 16 to 20, further comprising reducing, by the control device, the fluid flow rate of the at least one fluid injector device to provide a reduction in flow rate below the programmed fluid flow rate when a predetermined threshold pressure relative to the maximum pressure limit is reached.

Clause 22. The method of clause 21, wherein reducing the fluid flow rate is based on at least one input provided by a user of the fluid injector device.

Clause 23. The method of clause 22, wherein the at least one input provided by the user is provided for all fluid injection procedures performed by the fluid injector system, provided for each fluid injection procedure, or provided for a limited set of fluid injection procedures.

Clause 24. The method of clause 23, wherein the at least one user input provided by the user is selected by the user from a series of inputs saved in a protocol library or inputted manually by the user prior to initiation of the fluid injection procedure.

Clause 25. The method of clause 21, wherein reducing the fluid flow rate is based on at least one internally sourced or externally sourced input.

Clause 26. The method of clause 25, wherein the at least one internally sourced or externally sourced input is selected from the group consisting of historical injector data, historical patient data, information from one or more barcodes, one or more lookup tables, and combinations of any thereof, wherein the one or more barcodes is located on a patient record, the fluid injector, a medical fluid container, a sterile disposable, and combinations thereof.

Clause 27. The method of any one of clauses 17 to 26, wherein inputting or selecting the maximum fluid flow rate reduction input comprises inputting or selecting a user- or system-selectable input within a range of 0% to 100% of the programmed fluid flow rate.

Clause 28. The method of any one of clauses 17 to 27, further comprising aborting the injection procedure if a maximum fluid flow rate reduction input of 0% is selected and a pressure of the at least one fluid is detected to exceed the maximum pressure limit.

Clause 29. The method of any of clauses 17 to 28, wherein inputting or selecting the pressure limit sensitivity input comprises inputting or selecting a user- or system-selectable input within a configurable range from low sensitivity to high sensitivity.

Clause 30. The method of any one of clauses 21 to 28, further comprising aborting the injection procedure if specific configuration criteria are met and the pressure is detected to exceed the predetermined pressure threshold.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and the claims, the singular form of “a,” “an,” and “the” include plural referents unless context clearly dictates otherwise.

### BRIEF DESCRIPTION OF THE DRAWINGS

Additional advantages and details of the disclosure are explained in greater detail below with reference to the exemplary embodiments or aspects that are illustrated in the accompanying schematic figures, in which:

FIG. 1 is a perspective view of a fluid injector system according to one example of the present disclosure;

FIG. 2 is a perspective view of a multi-use disposable set for use with a fluid injector system of FIG. 1;

FIG. 3 is a perspective view of a fluid injector system according to another example of the present disclosure;

FIG. 4 is a schematic view of an electronic control system of a fluid injector system in accordance with examples of the present disclosure;

FIG. 5 is a graphical user interface of a fluid injector system according to one example of the present disclosure;

FIG. 6 is a graphical user interface of a fluid injector system according to another example of the present disclosure;

FIG. 7 is a graphical user interface of a fluid injector system according to another example of the present disclosure;

FIG. 8 is a graphical user interface of a fluid injector system according to another example of the present disclosure;

FIG. 9 is a graphical user interface of a fluid injector system according to another example of the present disclosure;

FIG. 10 is a graphical representation of configurable space under user- or system-defined maximum flow rate reduction settings in accordance with one example of the present disclosure;

FIG. 11 is a graphical representation of configurable space under user- or system-defined maximum flow rate reduction settings in accordance with another example of the present disclosure;

FIG. 12A is a pressure graph under a first user- or system-defined pressure limit sensitivity setting in accordance with one example of the present disclosure;

FIG. 12B is a pressure graph under a second user- or system-defined pressure limit sensitivity setting in accordance with another example of the present disclosure;

FIG. 12C is a pressure graph under a third user- or system-defined pressure limit sensitivity setting in accordance with another example of the present disclosure;

FIG. 12D is a pressure graph under a fourth user- or system-defined pressure limit sensitivity setting in accordance with another example of the present disclosure;

FIG. 13 is a flow chart representation of a fluid injection procedure according to one example of the present disclosure;

FIG. 14 is a graphical representation of corresponding pressure vs. time and flow rate vs. time graphs under user- or system-configured settings for a fluid injection procedure according to one example of the present disclosure;

FIG. 15 is a graphical representation of corresponding pressure vs. time and flow rate vs. time graphs under user- or system-configured settings for a fluid injection procedure according to another example of the present disclosure;

FIG. 16 is a graphical representation of corresponding pressure vs. time and flow rate vs. time graphs under user- or system-configured settings for a fluid injection procedure according to another example of the present disclosure;

FIG. 17 is a graphical representation of corresponding pressure vs. time and flow rate vs. time graphs under user- or system-configured settings for a fluid injection procedure according to another example of the present disclosure;

FIG. 18 is a graphical representation of corresponding pressure vs. time and flow rate vs. time graphs under user- or system-configured settings for a fluid injection procedure according to another example of the present disclosure;

FIG. 19 is a graphical representation of corresponding pressure vs. time and flow rate vs. time graphs under user- or system-configured settings for a fluid injection procedure according to another example of the present disclosure; and

FIG. 20 is a graphical representation of corresponding pressure vs. time and flow rate vs. time graphs under user- or system-configured settings for a fluid injection procedure according to another example of the present disclosure;

FIG. 21 illustrates a depiction on a graphical user interface of a sliding selector for adjusting the prioritization between limiting pressure and maintaining flow rate for user-configured settings for a fluid injection procedure according to an example of the present disclosure.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe of a multi-patient disposable set, the term “proximal” refers to a portion of a syringe nearest a piston for delivering fluid from a syringe.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a fluid reservoir, such as a syringe, a rolling diaphragm, or multiple syringe disposable set, the term “distal” refers to a portion of the fluid reservoir nearest to a patient. When used in relation to a fluid reservoir, such as a syringe, a rolling diaphragm, or multiple syringe disposable set, the term “proximal” refers to a portion of the fluid reservoir nearest to the injector system.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “server” may refer to one or more computing devices, such as processors, storage devices, and/or similar computer components that communicate with client devices and/or other computing devices over a network, such as the Internet or private networks, and, in some examples, facilitate communication among other servers and/or client devices. It will be appreciated that various other arrangements are possible. As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Non-limiting embodiments or aspects of the present disclosure are directed to systems, devices, products, apparatus, and/or methods for a fluid injector system that provides for improved pressure limiting behaviors. In particular, fluid injector systems provided herein may include computer algorithms and follow methods that allow a user or the system to utilize at least one of an internal input, for example an input by the system, or an external input, for example an input by a user or a hospital information network or other storage data device, to instruct at least one fluid injector device of the fluid injector system to perform a fluid injection procedure according to a profile that determines prioritization between maintaining a fluid flow rate and limiting a fluid delivery pressure. According to the present disclosure, the terms “flow rate” and “fluid flow rate” may be considered analogous to drug or contrast infusion rate, which can be altered by increasing or decreasing the overall flow of the drug or contrast containing solution. In other embodiments, flow rate or fluid flow rate may be altered by changing the concentration of the drug or contrast within the solution, for example by dilution with saline or addition of more concentrated solution of the drug or contrast agent, which can have the effect of changing the amount of drug/contrast delivered per volume delivered or can reduce the viscosity exponentially and drop the pressure of the fluid in the system due to reduced viscosity. It will be understood that the flow rate or fluid flow rate may be altered by various combinations of changing the overall flow of the solution or by altering the concentration and/or viscosity of the solution.

For example a depending on one or more of the inputs, the fluid delivery system may select or adapt a fluid injection protocol or injection profile that prioritizes maintaining flow rate or limiting a fluid delivery pressure and sets a predetermined pressure threshold and to abort the injection procedure if the pressure is detected, predicted, or trending to a equal or exceed a predetermined pressure threshold, where the predetermined pressure threshold is at least partially determined by the at least one of the internal input or an external input information.

According to various embodiments, the at least one of the internal input or an external input may include a maximum pressure limit for an injection procedure, a programmed fluid flow rate for injecting at least one fluid from the at least one fluid injector device, a maximum fluid flow rate reduction input for the injection procedure based off of at least one of patient information and system injection parameters, a pressure limit sensitivity input for the injection procedure, and various combinations of any thereof. For example, depending on the age of the patient and/or the overall health of the patient, including for example, the health of the vascular system of the patient, the user or system may prioritize maintaining flow rate or limiting a fluid delivery pressure. For example, in an older patient or a patient having poor vascular health (e.g., weak vascular walls), the user or system may prioritize limiting the fluid delivery pressure to avoid complications during the fluid delivery procedure. In another embodiment, where the patient may be the same age but have better vascular health, the prioritization may place more weight on maintaining flow rate and less on limiting fluid delivery pressure. In another embodiment, where the patient is healthy with a strong vascular system, the user or system may prioritize maintaining flow rate. As a result of the prioritization based on user or system input, the imaging procedure may provide an optimal fluid flow profile for the patient while minimizing potential hazards during the injection procedure.

User input may result from, but is not limited to, information based from patient examination, patient history data, user experience with patients of similar health or condition, type of prescribed injection procedure or protocol, time of injection, type of contrast, and various combinations thereof. System input may result from, but is not limited to, information based on historical injector data, historical patient data, information from one or more barcodes, one or more lookup tables, and combinations of any thereof. The one or more barcodes may be located on a patient record, the fluid injector, a medical fluid container, a sterile disposable, and combinations thereof, and may contain specific information on the article where the barcode is located. In various embodiments the inputted information may be a combination of user inputted information and system inputted information. Taking into account the inputted information, at least one control device may then develop an adaptive fluid flow for the injection profile that is specific for the patient and the particular fluid injector.

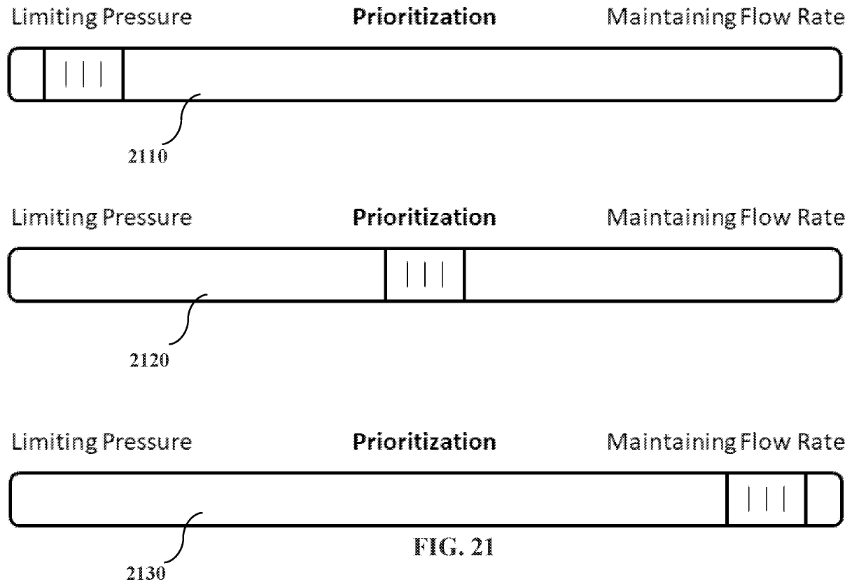

According to certain embodiments, the pressure limit sensitivity may range between a low sensitivity and a high sensitivity. In certain embodiments, the sensitivity of the pressure limit may have one or more intermediate sensitivities between low sensitivity and high sensitivity, such as an intermediate or “medium sensitivity”, depending on the desired fluid flow profile. According to other embodiments, the pressure limit sensitivity may be on a sliding scale between low sensitivity and high sensitivity. According to various embodiments, prioritization between pressure limit sensitivity and maintaining fluid flow rate may be at opposite ends of the scale as illustrated in FIG. 21. That is, when pressure limit sensitivity is of high priority (i.e., high pressure limit sensitivity), then maintaining fluid flow rate may be of low priority; and when pressure limit sensitivity is of low priority (i.e., low pressure limit sensitivity), then maintaining fluid flow rate may be of high priority.

According to various embodiments inputting the at least one of the internal input or an external input and/or changing the prioritization between pressure limit sensitivity and maintaining fluid flow rate may be done prior to starting the injection procedure or can be done during the execution of the injection procedure. The injection protocol may be updated based on any of the before-injection data and/or based on real-time injection data being collected during the injection procedure. According to certain embodiments, the determination of the allowable injection profile may be weighted to provide priority to pre-injection information over real-time injection information or vice versa. Further, the injection information may include, for example, pressure, fluid flow rate, scanner data or image feedback, patient physiological feedback (e.g., ECG signal, blood pressure, heart rate, body temperature) or combinations thereof.

Various injection parameters that may be inputted, by the user and/or the fluid injector system, to at least partially prioritize between pressure limitation and maintaining flow rate include but are not limited to: historical data from the system or the user; data fed into the system from the user, an external system (picture archiving and communications systems (PACS), radiology information systems (RIS), hospital information systems (HIS), external medical records, etc.), data from barcodes, RFID tags, and other near-field communication tags, such as tags on injector disposables, contrast media and medical fluid containers, catheters, patient wristband etc.; one or more lookup table on various parameters for specific injection protocols, e.g., stored within the memory of the control device, a network server, or hard copy versions available to user, among others. According to various embodiments, the parameters may be configured previously as part of each individual protocol, the parameters may be received from the scanner as part of the patient procedure information or other data packet, the parameters may be otherwise configured to automatically change or update based on the combination of injection protocol and disposable (e.g., syringe size, catheter size, fluid type. etc.), or various combinations of parameters received from these sources.

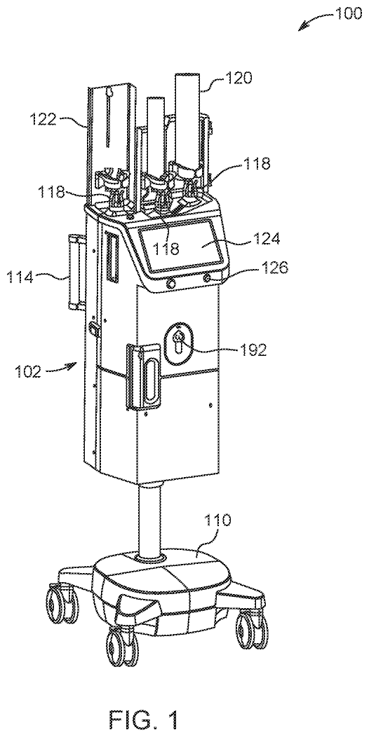

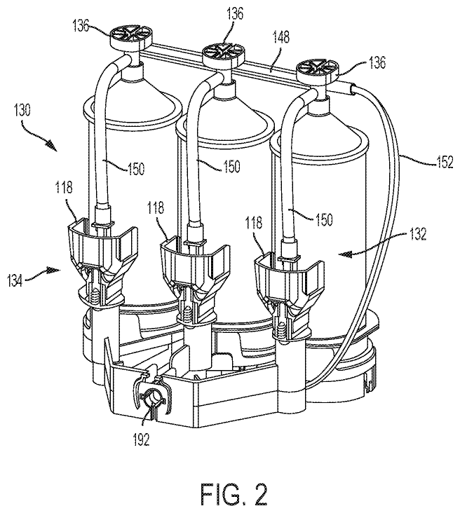

Referring to the drawings in which like reference characters refer to like parts throughout the several views thereof, one aspect or example of the present disclosure is generally directed to a multi-fluid medical injector/injector system 100 (hereinafter “fluid injector system 100”) which in certain embodiments may include a multi-use disposable set (MUDS) 130 configured for delivering fluid to a patient using a single-use disposable set (SUDS) connector (not shown) and in other embodiments may include two or more disposable fluid reservoirs or syringes, which may be disposed after one injection procedure or a specific number of injection procedures. The fluid injector system 100 may include multiple components as individually described herein. Generally, the fluid injector system 100 depicted in FIGS. 1-2 has a powered injector or other administration device and a fluid delivery set intended to be associated with the injector to deliver one or more fluids from one or more multi-dose containers under pressure into a patient, as described herein. The various devices, components, and features of the fluid injector system 100 and the fluid delivery set associated therewith are likewise described in detail herein. While the various examples of the methods and processes are shown with reference to an injector system having a multi-use disposable set (“MUDS”) and a single-use disposable set (“SUDS”) configuration in FIGS. 1-2, the disclosure is not limited to such an injector system and may be utilized in other syringe based injector systems, such as but not limited to those described in U.S. Pat. Nos. 7,553,294, 7,563,249, 8,945,051, 9,173,995, 10,124,110; 10,507,319; and 10,583,256; and U.S. application Ser. No. 15/568,505; the disclosures of each of which are incorporated herein in their entirety by this reference.

With reference to FIG. 1, a fluid injector system 100 according to one example includes an injector housing 102 that encloses the various mechanical drive components, electrical and power components necessary to drive the mechanical drive components, and control components, such as electronic memory and electronic control devices, used to control operation of reciprocally movable pistons (not shown) associated with the fluid injector system 100 described herein. Such pistons may be reciprocally operable via electro-mechanical drive components such as a ball screw shaft driven by a motor, a voice coil actuator, a rack-and-pinion gear drive, a linear motor, and the like.

The fluid injector system 100 may include at least one bulk fluid connector 118 for connection with at least one bulk fluid source 120. In some examples, a plurality of bulk fluid connectors 118 may be provided. For example, as shown in the fluid injector embodiment illustrated in FIG. 1, three bulk fluid connectors 118 may be provided in a side-by-side or other arrangement. In some examples, the at least one bulk fluid connector 118 may include a spike configured for removably connecting to the at least one bulk fluid source 120, such as a vial, a bottle, or a bag. The at least one bulk fluid connector 118 may be formed on the multi-use disposable set (“MUDS”), as described herein. The at least one bulk fluid source 120 may be configured for receiving a medical fluid, such as saline, Ringer's lactate, an imaging contrast medium solution, or other medical fluid, for delivery to the patient by the fluid injector system 100.

With reference to FIG. 2, a MUDS 130 is configured for being removably connected to the fluid injector system 100 for delivering one or more fluids from the one or more bulk fluid sources 120 to the patient. Examples and features of embodiments of the MUDS are further described in PCT International Publication No. WO 2016/112163, filed on Jan. 7, 2016, the disclosure of which is incorporated herein by reference in its entirety. The MUDS 130 may include one or more fluid reservoirs, such as one or more syringes 132. As used herein, the term “fluid reservoir” means any container capable of taking in and delivering a fluid, for example during a fluid injection procedure including, for example a syringe, a rolling diaphragm, a pump, a compressible bag, and the like. Fluid reservoirs may include the interior volume of at least a portion of a fluid pathway, such as one or more tubing lengths, that are in fluid communication with the interior of the fluid reservoir, including fluid pathway portions that remain in fluid communication with the fluid reservoir after the system is closed or fluidly isolated from the remainder of the fluid pathway. In some examples, the number of fluid reservoirs may correspond to the number of bulk fluid sources 120 (shown in FIG. 1). For example, with reference to FIG. 2, the MUDS 130 has three syringes 132 in a side-by-side arrangement such that each syringe 132 is fluidly connectable to one or more of the corresponding three bulk fluid sources 120. In some examples, one or more bulk fluid sources 120 may be connected to one or more syringes 132 of the MUDS 130. Each syringe 132 may be fluidly connectable to one of the bulk fluid sources 120 by a corresponding bulk fluid connector 118 and an associated MUDS fluid path 134. The MUDS fluid path 134 may have a spike element that connects to the bulk fluid connector 118 and the fluid line 150. In some examples, the bulk fluid connector 118 may be provided directly on MUDS 130.

With continued reference to FIGS. 1 and 2, the MUDS 130 may include one or more valves 136, such as stopcock valves, for controlling which medical fluid or combinations of medical fluids are withdrawn from the multi-dose bulk fluid source 120 (see FIG. 1) into the fluid reservoirs 132 and/or are delivered to a patient from each fluid reservoir 132. In some examples, the one or more valves 136 may be provided on a distal end of the plurality of syringes 132 or on a manifold 148. The manifold 148 may be in selectable fluid communication via valves 136 with the interior volume of the syringes 132. The interior volume of the syringes 132 may be in selectable fluid communication via valves 136 with a first end of the MUDS fluid path 134 that connects each syringe 132 to the corresponding bulk fluid source 120. The opposing second end of the MUDS fluid path 134 may be connected to the respective bulk fluid connector 118 that is configured for fluidly connecting with the bulk fluid source 120. Depending on the position of the one or more valves 136, fluid may be drawn into the interior volume of the one or more syringes 132 or it may be delivered from the interior volume of the one or more syringes 132. In a first position, such as during the filling of the syringes 132, the one or more valves 136 are oriented such that fluid flows from the bulk fluid source 120 into the desired syringe 132 through a fluid inlet line 150, such as a MUDS fluid path. During the filling procedure, the one or more valves 136 are positioned such that fluid flow through one or more fluid outlet lines 152 or manifold 148 is blocked or closed. In a second position, such as during a fluid delivery procedure, fluid from one or more syringes 132 is delivered to the manifold 148 through the one or more fluid outlet lines 152 or syringe valve outlet ports. During the delivery procedure, the one or more valves 136 are positioned such that fluid flow through one or more fluid inlet lines 150 is blocked or closed. In a third position, the one or more valves 136 are oriented such that fluid flow through the one or more fluid inlet lines 150 and the one or more fluid outlet lines 152 or manifold 148 is blocked or closed. Thus, in the third position, each of the one or more valves 136 isolates the corresponding syringe 132 and prevents fluid flow into and out of the interior volume of the corresponding syringe 132. As such, each of the one or more syringes 132 and the corresponding valve 136 defines a closed system.

The one or more valves 136, fluid inlet lines 150, and/or fluid outlet lines 152 may be integrated into or in fluid communication via the manifold 148. The one or more valves 136 may be selectively positioned to the first or second position by manual or automatic handling. For example, the operator may position the one or more valves 136 into the desired position for filling, fluid delivery, or the closed position. In other examples, at least a portion of the fluid injector system 100 is operable for automatically positioning the one or more valves 136 into a desired position for filling, fluid delivery, or the closed position based on input by the operator or by a protocol executed by the electronic control unit.

With continued reference to FIGS. 1 and 2, according to the described embodiment the fluid injector system 100 may have a connection port 192 that is configured to form a releasable fluid connection with at least a portion of the SUDS. In some examples, the connection port 192 may be formed on the MUDS 130. As described herein, the SUDS may be connected to the connection port 192, formed on at least a portion of the MUDS 130 and/or the housing 102. Desirably, the connection between the SUDS and the connection port 192 is a releasable connection to allow the SUDS to be selectively connected to and disconnected from the connection port 192. In some examples, the SUDS may be disconnected from the connection port 192 and disposed after each fluid delivery procedure, and a new SUDS may be connected to the connection port 192 for a subsequent fluid delivery procedure. The SUDS may be used to deliver one or more medical fluids to a patient by SUDS fluid line having a distal end that may be selectively disconnected from the body of the SUDS and connected to a patient catheter. Other examples and features of the SUDS are described in U.S. Patent Publication No. 2016/0331951, filed Jul. 7, 2016, the disclosure of which is incorporated herein by reference in its entirety.

Referring again to FIG. 1, the fluid injector system 100 may include one or more user interfaces 124, such as a graphical user interface (GUI) display window. The user interface 124 may display information pertinent to a fluid injection procedure involving fluid injector system 100, such as injection status or progress, current flow rate, fluid pressure, and volume remaining in the at least one bulk fluid source 120 connected to the fluid injector system 100 and may be a touch screen GUI that allows an operator to input commands and/or data for operation of fluid injector system 100. Additionally, the fluid injector system 100 and/or user interface 124 may include at least one control button 126 for tactile operation by an attendant operator of the fluid injector system 100. The at least one control button 126 may be a graphical part of the user interface 124, such as a touch screen.

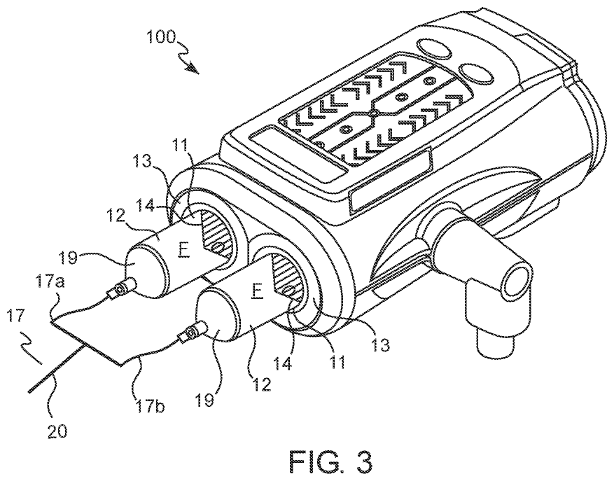

While FIGS. 1-2 illustrate one example of a fluid injector system 100 and associated components and structure, it is to be understood that the present disclosure is not limited to any particular type or variety of the fluid injector system 100. Referring now to FIG. 3, another non-limiting example of a fluid injector system 100 in accordance with the present disclosure includes at least one fluid reservoir, such as syringe 12, at least one piston (not pictured) connectable to at least one plunger 14, and a fluid control module (not pictured). The at least one syringe 12 is generally adapted to interface with at least one component of the system, such as a syringe port 13. The fluid injector system 100 is generally configured to deliver at least one fluid F to a patient during an injection procedure. The fluid injector system 100 is configured to releasably receive the at least one syringe 12, which is to be filled with at least one fluid F, such as a contrast media, saline solution, Ringer's lactate, or any desired medical fluid. The system may be a multi-syringe injector, wherein several syringes may be oriented side-by-side or in another spatial relationship and are separately actuated by respective pistons associated with the injector. The at least one syringe 12 may be oriented in any manner such as upright, downright, or positioned at any degree angle. In another embodiment, a fluid injector 100 may interface with one or more rolling diaphragm syringes (not shown). Non-limiting examples of rolling diaphragm syringe based injectors are described in U.S. application Ser. Nos. 15/305,285, and 15/568,505 and PCT International Application No. PCT/US2017/056747, the disclosures of which are incorporated herein.

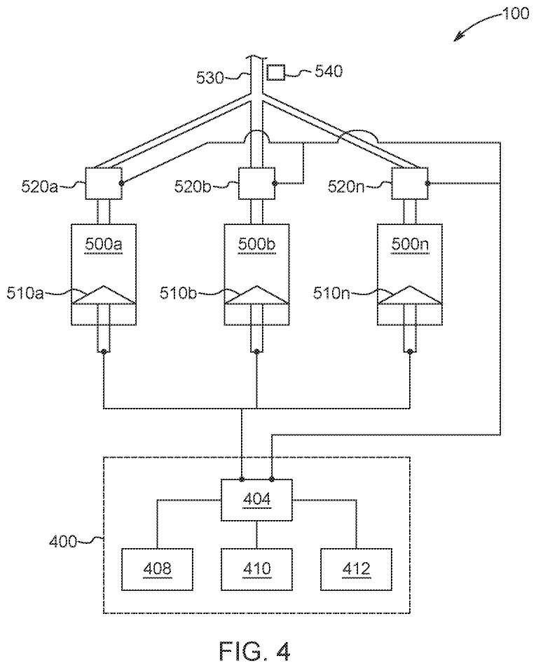

With continued reference to FIG. 3, the injector system 100 may be used during a medical procedure to inject the at least one medical fluid F into the vasculature system of a patient by driving a plunger 14 of at least one syringe 12 with a drive member, such as the at least one piston 103 (see FIG. 4). The at least one piston may be reciprocally operable upon at least a portion of the at least one syringe, such as the plunger 14. Upon engagement, the at least one piston may move the plunger 14 toward the distal end 19 of the at least one syringe, as well as retracting the plunger 14 toward the proximal end 11 of the at least one syringe 12.

A tubing set 17 (e.g., first and second fluid conduits 17a and 17b, and common fluid conduit 20) may be in fluid communication with an outlet port of each syringe 12 to place each syringe in fluid communication with a catheter for delivering the fluid F from each syringes 12 to the catheter (not shown) inserted into a patient at a vascular access site. The first and second fluid conduits 17a and 17b may be connected to the common fluid conduit 20 by any suitable mechanism known in the art (e.g., a Y-connector or a T-connector). The fluid injector system 100 shown in FIG. 3 is an open system do to the lack of valves capable of isolating the syringes 12 from one another and from at least a portion of the tubing set 17. However, it is to be understood that valves, similar or identical to the valves 136 described with reference to the fluid injector system 100 of FIGS. 1 and 2, may be added distally of the syringes 12 to convert the fluid injector system 100 of FIG. 3 to a closed system.

Referring now to FIG. 4, fluid injector systems 100 in accordance with the present disclosure may be associated with and controlled by an electronic control device 400 configured to execute one or more injector protocols including, for example, the filling, priming, and delivery operations. In some examples, the electronic control device 400 may control the operation of various valves, stopcocks, piston members, and other elements to affect a desired gas/air removal, filling, and/or delivery procedure. The electronic control device 400 may include at least one processor 404, memory 408, an input component 410, and an output component 412. The electronic control device further may include a bus that permits communication among the components of electronic control device 400. The at least one processor 404 may be implemented in hardware, firmware, or a combination of hardware and software. For example, processor 404 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 408 may include a hard disk (e.g., a magnetic disk, an optical disk, a magneto-optic disk, a solid-state disk, etc.) and/or another type of computer-readable medium. The input component 410 may include a component that permits the electronic control device 400 to receive information, such as via user input (e.g., the user interface 124). The output component 412 may include a component that provides output information from the electronic control device 400 (e.g., the user interface 124).

The electronic control device 400 may be programmed or configured to perform one or more processes and/or methods based on the at least one processor 404 executing software instructions stored by a computer-readable medium, such as memory 408. When executed, software instructions stored in memory 408 may cause the at least one processor 404 to perform one or more processes and/or methods described herein.

With continued reference to FIG. 4, the electronic control device 400, more particularly the at least one processor 404, may be in operative communication with one or more components of the fluid injector system 100 to control an operation of the fluid injector system 100. The electronic control device 400 may be in operative communication with one or more drive components 510a, 510b, 510n respectively associated with one or more fluid reservoirs 500a, 500b, 500n of the fluid injector system 100 to control filling of fluid and delivery of fluid from the fluid reservoirs 500a, 500b, 500n. More particularly, each of the one or more drive components 510a, 510b, 510n may be associated with one of the fluid reservoirs 500a, 500b, 500n such that fluid contained in each of the fluid reservoirs 500a, 500b, 500n may be selectively delivered via actuation of the associated drive component 510a, 510b, 510n. The fluid reservoirs 500a, 500b, 500n may be, or may correspond to, the syringes 132 of the fluid injector system 100 of FIGS. 1-2 and/or the syringes 12 of the fluid injector system 100 of FIG. 3 or other syringe-type structures, such as rolling diaphragm syringes, as described herein. The one or more drive components 510a, 510b, 510n may be, or may correspond to, the pistons (not pictured) of the fluid injector systems 100 of FIGS. 1-3. The one or more fluid reservoirs 500a, 500b, 500n may be in fluid communication with a fluid conduit 530 for delivering fluid to a catheter or other component connected to a patient. The fluid conduit 530 may be, or may correspond to, the SUDS of the fluid injector system 100 of FIGS. 1-2 and/or the tubing set 17 of the fluid injector system 100 of FIG. 3

In aspects and examples of a closed fluid injector system 100 (e.g., the fluid injector system 100 of FIGS. 1 and 2), the electronic control device 400 further may be in operative communication with one or more valves 520a, 520b, 520n in order to rotate or otherwise actuate the valves 520a, 520b, 520n to direct flow into or out of and/or isolate flow from one or more of the fluid reservoirs 500a, 500b, 500n to the fluid conduit 530. The valves 520a, 520b, 520n may be, or may correspond to, the valves 136 described herein in connection with FIG. 2.

As noted above, conventional fluid injector systems utilize pressure limit settings that are determined by the manufacturer and hard-coded into the system. However, in order to support a diverse patient population, a variety of injection protocols, and multiple IV sites/types, there is a need for user- or system-configurable and/or customizable methods of setting pressure limiting behavior. As such, and in accordance with an aspect of the disclosure, a fluid injection system and method for providing user- or system-configurable injector performance settings is disclosed.

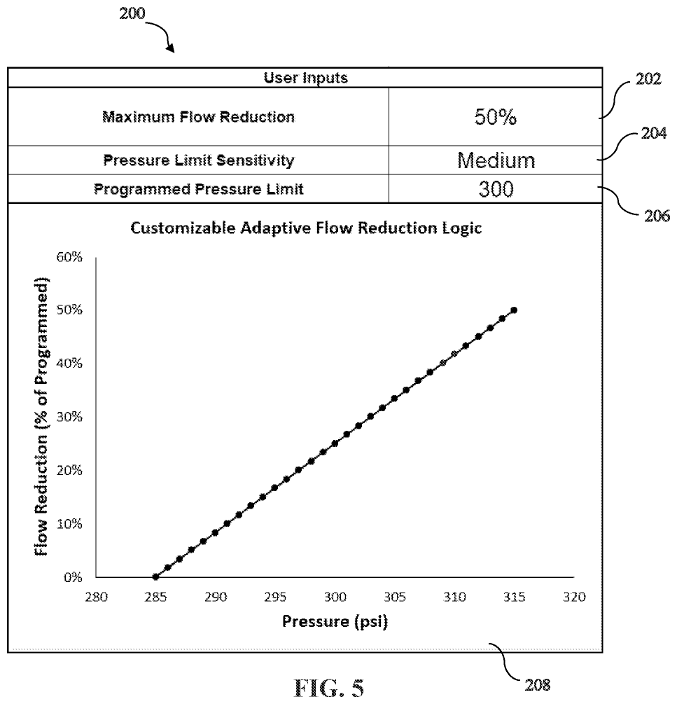

Referring to FIGS. 5-9, a graphical user interface (GUI) 200 in accordance with various embodiments of the disclosure is illustrated. While only one GUI 200 is illustrated, it is to be understood that more than one GUI 200 may be present. GUI 200 may be a touch-screen interface located on, e.g., the user interface 124 of fluid injector system 100, as shown in FIG. 1. However, it is to be understood that GUI 200 is not limited as such and may be located remotely from the fluid injector system 100. GUI 200 includes at least two user- or system-configurable inputs: a maximum flow reduction input 202 and a pressure limit sensitivity input 204. Based on the settings selected by the medical practitioner for each of these inputs, customizable injector behavior is achievable, with the selections being made dependent upon whether maintaining flow rate (i.e., contrast/iodine delivery rate, or IDR) or ensuring that the pressure limit is never breached is of greater importance given the variables of the injection procedure.

In addition to the at least two user- or system-configurable inputs, the GUI 200 may also include a programmed pressure limit indicator 206 and an adaptive flow graph 208. The pressure limit identified on the programmed pressure limit indicator 206 is typically pre-programmed based on the type of injection procedure. For example, for an injection procedure related to CT imaging, the programmed pressure limit may be 300 psi, while for an angiography procedure, the programmed pressure limit may be 1200 psi. In some embodiments, the programmed pressure limit may be input via the GUI 200. As will be described in further detail below, the adaptive flow graph 208 provides the practitioner with a visual indication of the user- or system-customized injector settings relative to one another. However, it is to be understood that the adaptive flow graph 208 may be omitted from the GUI 200, with only the user- or system-configurable inputs being shown.

Referring to FIG. 5, GUI 200 in accordance with a first setting configuration is illustrated. As is shown in this example configuration, the maximum flow reduction input 202 is set to 50%, while the pressure limit sensitivity 204 is set to “Medium”. Additionally, the programmed pressure limit indicator 206 indicates a pressure limit of 300 psi for the subject injection procedure.

Regarding the maximum flow reduction input 202, the practitioner is able to select the flow rate reduction within a configurable range of 0% to 50% of an originally programmed (i.e., commanded) flow rate. For example, according to a non-limiting embodiment, if the maximum flow reduction input 202 is set to 50% (as shown in FIG. 5), and the programmed flow rate is 5 mL/s, the flow rate will be reduced to no lower than 2.5 mL/s as the injector system is operating at or near the programmed pressure limit. Likewise, if the maximum flow reduction input 202 is set to 40%, and the programmed flow rate is 5 mL/s, the flow rate will be reduced to no lower than 3 mL/s at or near the programmed pressure limit. Thus, configuring a lower maximum flow reduction input 202 will ensure that the injection system prioritizes maintaining fluid flow rate, even as the programmed pressure limit is approached and/or reached. In this way, injection procedures where pressure sensitivity is of lesser importance than sufficient fluid flow rate may be accounted for through user- or system-configurable settings prioritizing low flow reduction percentages over pressure limit sensitivity.

In the event that the pressure dictates that the minimum flow rate cannot be achieved, the system can be configured to abort the injection procedure entirely. Similarly, if the user configures the maximum flow rate reduction input 202 to be 0% (i.e., no permissible change in flow rate), the injection can be aborted if the pressure is ever detected to exceed the programmed pressure limit.

While a configurable flow reduction range of 0% to 50% is provided in the examples illustrated herein, it is to be understood that the configurable range of maximum flow reduction input 202 may be lesser or greater than 0% to 50% and need not necessarily be a percentage-based range. The range can be narrowed dependent upon the imaging modality, with the acceptable reduction in flow rate being determined based upon a flow rate at which an acquired image would still be considered sufficient for diagnostics. For example, in CT imaging, the configurable flow reduction range may be 0% to 50%. In other imaging modalities where flow rate is less crucial to a successful imaging procedure, the configurable flow range may be, e.g., from 0-100%, where 100% means the injection is aborted completely.

Referring to the pressure limit sensitivity input 204 in FIG. 5, the “Medium” setting may be selected from an incremental configurable range extending from, e.g., “Low,” “Medium/high,” to “High”. In this configuration, a selection of “High” sensitivity dictates that the injection system ensures that the pressure does not exceed the programmed pressure limit or threshold. Such a pressure limit sensitivity selection may be particularly applicable to injection procedures performed at sensitive injection sites or on sensitive patients, where remaining below the pressure ratings of all components of the system is of the utmost importance. Conversely, a selection of “Low” sensitivity provides the system with greater flexibility to maintain a programmed flow rate while still limiting pressure from greatly exceeding the programmed pressure limit or threshold. The “Medium” setting, as shown in FIG. 5, provides a compromise between the “High” and “Low” sensitivity selections. Additional incremental sensitivity settings that may be selected by a user (e.g., “Low/Medium”, “Medium/High”, etc.) provide for more nuanced sensitivities between “Low” and “Medium” or between “Medium” and “High”. Furthermore, it is to be understood that the pressure limit sensitivity input 204 is not limited to these selections, and that numerical sensitivity ranges (e.g., 0-to-10) or other types of incremental ranges may be implemented. With the “High” (or maximum) sensitivity setting, a maximum allowable flow rate reduction is achieved before or when an initial pressure limit or threshold is reached. Conversely, with the “Low” (or minimum) sensitivity setting, a flow rate reduction may be first initiated when the initial pressure limit or threshold is reached.

According to specific embodiments when considering the range of responsiveness to increasing pressure (i.e., “Low” to “Medium” to “High”) the range may be set such that: “Low” may range from 15% to 35% of the maximum responsiveness, for example in one embodiment, “Low” may be 25%; “Medium” may range from 40% to 60% of the maximum responsiveness, for example in one embodiment, “Medium” may be 50%; and “High” may range from 65% to 85% of the maximum responsiveness, for example in one embodiment, “High” may be 75%. “Low/Medium” and “Medium/High” may be then be a similar range between “Low” and “Medium” and between “Medium” and “High”, respectively. According to other embodiments, the range of responsiveness may be determined by inputs such as maximum flow rate reduction; or may be hard-coded values depending on the system configuration, modality within which the device is being used, clinical settings, patient settings, etc. These numbers can all change as desired. For example, in one embodiment, “Low” may be 90%, “Medium” may be 95%, and “High” may be 100% of the maximum responsiveness. One of skill in the art can envision other set ranges for “Low”, “Medium”, and “High” according to other embodiments.

The adaptive flow graph 208 provides for a visual illustration of the injector behavior based on the user- or system-selected settings of the maximum flow rate reduction input 202 and the pressure limit sensitivity input 204. In the example shown in FIG. 5, it is to be understood that the commanded flow rate is maintained until the measured pressure reaches 285 psi, or 95% of the programmed pressure limit. According to various embodiments, the thresholds may vary depending on system configuration, modality, injection speed, syringe and/or tubing size, catheter size or configuration, patient information, and combinations of any thereof. Once this threshold is reached, the flow rate is reduced by as much as 50% of the commanded flow rate, even as the pressure rises above the programmed pressure limit of 300 psi (i.e., to 315 psi, or 105% of the programmed pressure limit). The reduction in flow rate may be based on a predetermined flow reduction profile, which may be linear or non-linear (e.g., polynomial, exponential, logarithmic, etc.). This predetermined flow rate reduction and allowance for slight overpressure is made possible by the user- or system-configured “Medium” pressure limit sensitivity input 204. Additionally, the predetermined and wide-ranging flow reduction from 0% to 50% of commanded flow rate is made possible by the user- or system-configured “50%” maximum flow reduction input 202.

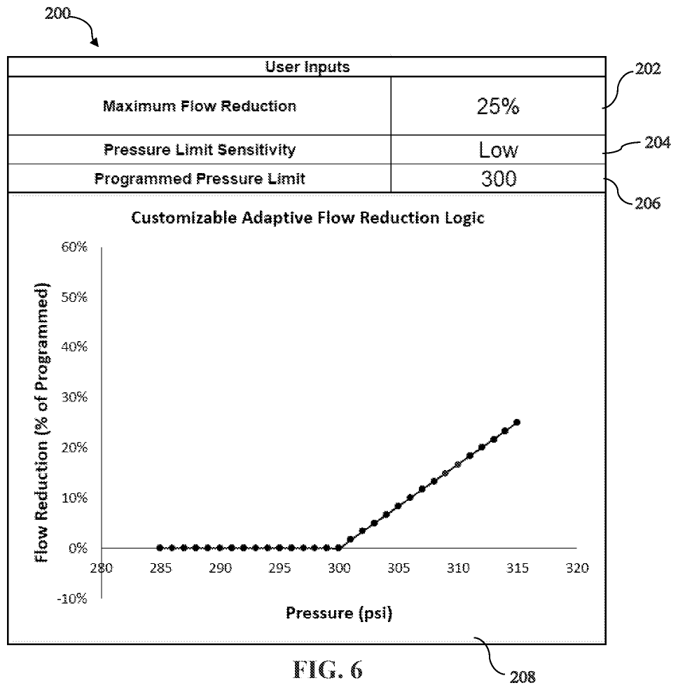

Referring to FIGS. 6-9, additional examples of GUI 200 under varying user- or system-defined configurations are shown. In FIG. 6, the maximum flow rate reduction input 202 is set to 25%, and the pressure limit sensitivity input 204 is set to “Low”. Accordingly, as explained above, the injection procedure illustrated in FIG. 6 prioritizes maintaining the fluid flow rate over not exceeding the programmed pressure limit. Such is easily discernable by the adaptive flow graph 208 in FIG. 6, which illustrates that under a “Low” pressure limit sensitivity, the commanded flow rate is actually maintained up until the point that the programmed pressure limit is reached (i.e., a pressure of 300 psi). Then, after the programmed pressure limit is reached, the flow rate is reduced based on a linear or non-linear predetermined flow reduction profile, but only to an amount of 25% of the commanded flow rate. In this way, the fluid flow rate is substantially maintained, even after reaching the programmed pressure limit.

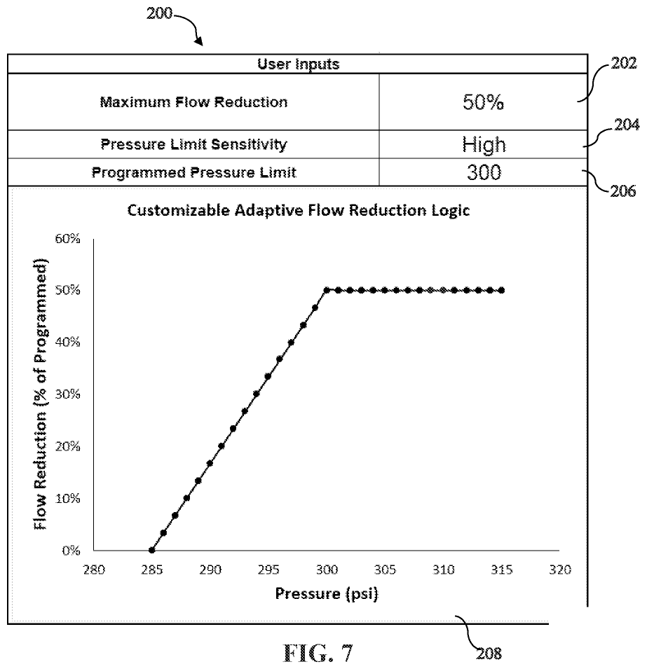

Conversely, FIG. 7 illustrates an injection procedure in which sensitivity to the pressure limit is prioritized over maintenance of the flow rate. As is shown, the maximum flow rate reduction input 202 is set to 50%, and the pressure limit sensitivity input 204 is set to “High”. Referring to the adaptive flow graph 208 in FIG. 7, under a “High” pressure limit sensitivity, all reductions in flow rate occur prior to the point at which that the programmed pressure limit (i.e., 300 psi) is reached in an attempt to ensure that the programmed pressure limit is not exceeded. Then, after the programmed pressure limit is reached, the reduction in flow rate is maintained at the maximum level (i.e., 50%) in an effort to reduce the pressure below the predefined pressure limit. In some embodiments, when a “High” pressure limit sensitivity is selected, the injection system may be configured to abort the fluid injection if and when the pressure is ever detected to equal or exceed the programmed pressure limit. In other embodiments, the injection system may be configured to abort the fluid injection if and when the pressure is ever detected to equal or exceed a predetermined secondary pressure threshold that is different from the programmed pressure limit.

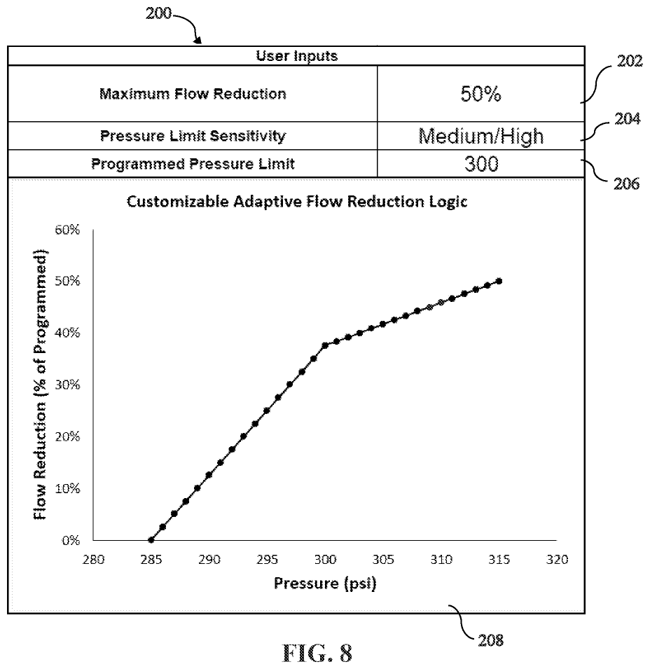

FIG. 8 illustrates an injection procedure in which sensitivity to the pressure limit is slightly prioritized over maintenance of the flow rate. As is shown, the maximum flow rate reduction input 202 is set to 50%, and the pressure limit sensitivity input 204 is set to “Medium/High”. Referring to the adaptive flow graph 208 in FIG. 8, under a “Medium/High” pressure limit sensitivity, all reductions in flow rate occur prior to the point at which that the programmed pressure limit (i.e., 300 psi) is reached in an attempt to ensure that the programmed pressure limit is not exceeded. Then, after the programmed pressure limit (300 psi at approx. 38% flow reduction) is reached, the reduction in flow rate is maintained at the medium level in an effort to reduce the pressure below the predefined pressure limit. In some embodiments, when a “Medium/High” pressure limit sensitivity is selected, the injection system may be configured to abort the fluid injection if and when the pressure is ever detected to equal or exceed the programmed pressure limit. In other embodiments, the injection system may be configured to abort the fluid injection if and when the pressure is ever detected to equal or exceed a predetermined secondary pressure threshold that is different from the programmed pressure limit.

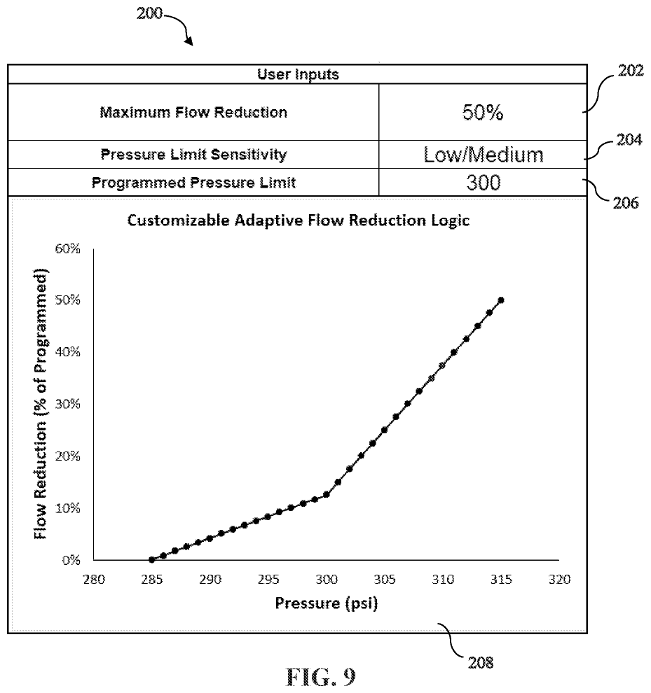

FIG. 9 illustrates an injection procedure in which maintenance of the flow rate is slightly prioritized over sensitivity to the pressure limit. As is shown, the maximum flow rate reduction input 202 is set to 50%, and the pressure limit sensitivity input 204 is set to “Low/Medium”. Referring to the adaptive flow graph 208 in FIG. 9, under a “Low/Medium” pressure limit sensitivity, after the programmed pressure limit (i.e., 300 psi) is reached after the programmed pressure limit is reached, the flow rate is reduced based on a linear or non-linear predetermined flow reduction profile, at an amount of 50% of the commanded flow rate. In this way, the fluid flow rate is substantially maintained, even after reaching the programmed pressure limit., and the reduction in flow rate is maintained at the low level. In some embodiments, when a “Low/Medium” pressure limit sensitivity is selected, the injection system may be configured to abort the fluid injection if and when the pressure is ever detected to equal or exceed the programmed pressure limit.

While FIGS. 5-9 provide examples of GUI 200 where one of the maximum flow rate reduction input 202 and the pressure limit sensitivity input 204 is set to a relatively “extreme” setting (i.e., a “High” pressure limit sensitivity, a 50% maximum flow rate reduction, etc.), it is to be understood that under any injection protocols, such extreme settings may not be necessary, and a more moderate settings for both inputs may be optimal. In fact, in some embodiments, the injector system may include default logic set by the manufacturer, wherein the injection protocol is set for moderate flow rate reduction and moderate pressure limit sensitivity.

Additionally, while the of the maximum flow rate reduction input 202 and the pressure limit sensitivity input 204 are shown and described herein as being input via the touchscreen GUI 200, it is to be understood that other forms of user interfaces may be utilized to provide such inputs. For example, the user interface may include a keyboard, a mouse, one or more buttons, one or more knobs, etc. Furthermore, as noted above, the user interface may be integrated into the fluid injector system 100 or it may be located remotely from fluid injector system 100. If located remotely from fluid injector system 100, the user interface may be capable of wired or wireless communication with the electronic control device 400. Referring to FIG. 21, an embodiment is illustrated for a GUI 200 having a user adjustable sliding scale between prioritization of limiting the pressure 2110 and maintaining fluid flow rate 2130, including an intermediate protocol 2120. The GUI 200 may be a touchscreen that allows a user to adjust prioritization by sliding their finger along the scale to the desired prioritization. Alternatively, the sliding scale may be adjusted by a keyboard, a mouse, one or more buttons, or one or more knobs associated with the GUI and controller.

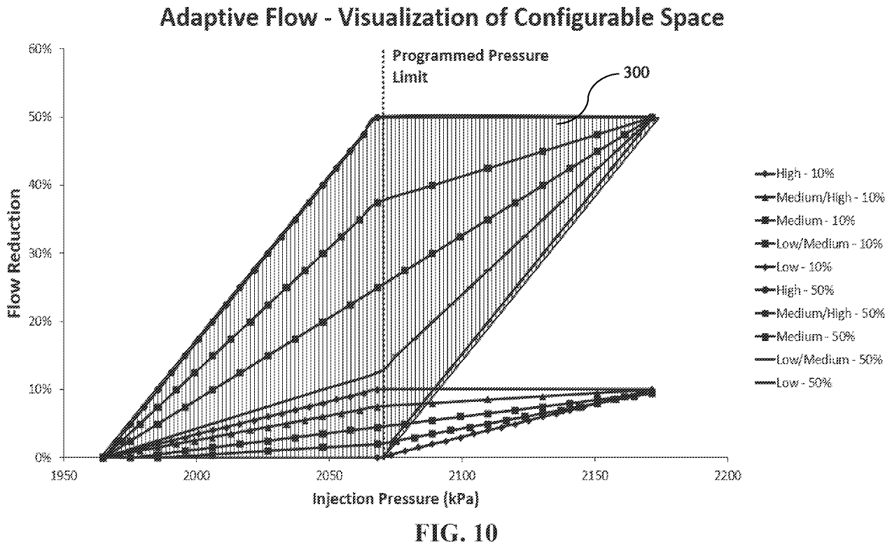

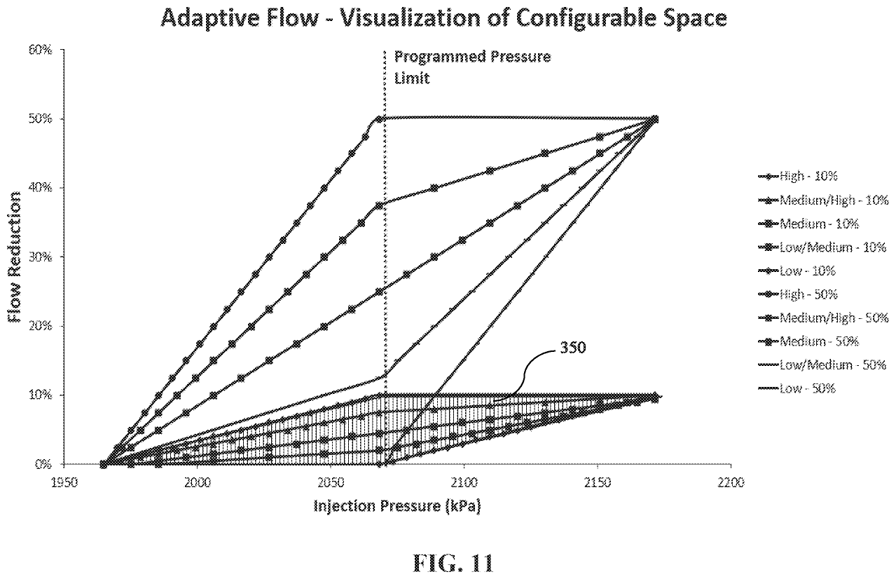

Referring now to FIGS. 10-11, graphical visualizations of the configurable space under various user- or system-selected flow behavior settings are illustrated. In FIG. 10, the shaded area 300 represents the possible configurable space in which a maximum flow rate reduction input of 50% is selected. With such an expansive range of flow rate reduction, the shaded area 300 illustrates the various possible user- or system-selectable configurations, from a “High” pressure limit sensitivity, where all flow rate reduction takes place prior to the programmed pressure limit being reached, to a “Low” pressure limit sensitivity, where all flow rate reduction takes place after the programmed pressure limit is reached.

In FIG. 11 is similar to FIG. 10, but the shaded area 350 represents the possible configurable space in which a maximum flow rate reduction input of 10% is selected. As is easily discernable from FIG. 11, reducing the maximum flow rate reduction input provides for a much smaller configurable space at or near the programmed pressure limit, which may be advantageous when maintaining flow rate is to be prioritized. While not shown, it is to be understood that similar graphical visualizations could be generated for any maximum flow rate reduction input (i.e., any input between 0% and 50%).

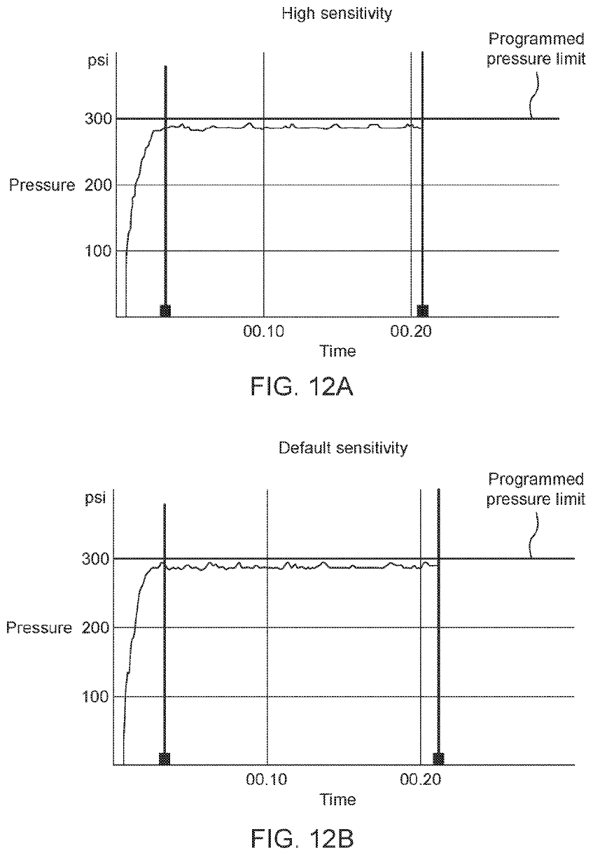

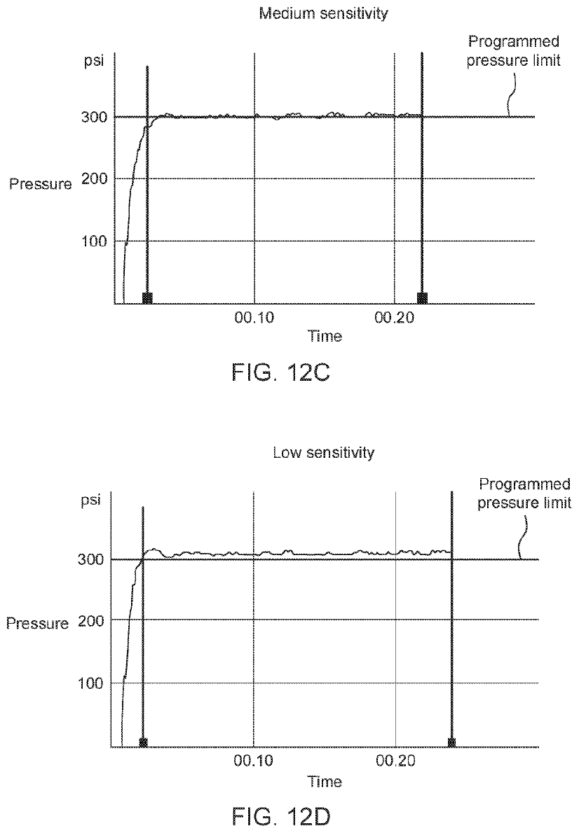

Next, FIGS. 12A-12D illustrate expected differences in a pressure graph for a particular injection (e.g., a 5 mL/s contrast injection) under various user- or system-selected configurations for pressure limit sensitivity.

First, FIG. 12A illustrates a “High” pressure limit sensitivity setting. As can be seen, under this “High” sensitivity setting, the system pressure may gradually approach the programmed pressure limit (e.g., 300 psi), but the flow rate is reduced a sufficient amount such that there is substantial leeway between the observed system pressure and the pressure limit. That is, the pressure limit of 300 psi is never reached.

FIG. 12B illustrates a “Default” pressure limit sensitivity setting. Unlike the “High” setting described above with respect to FIG. 12A, the “Default” setting may allow the system pressure to more closely approach (and perhaps even reach) the programmed pressure limit.

As discussed above, under such a “Default” setting, the system may allow for a moderate amount of flow reduction, as well as a moderate pressure sensitivity.

Referring to FIG. 12C, a “Medium” pressure limit sensitivity is shown, wherein the system pressure is actually allowed to slightly exceed the programmed pressure limit. As was similarly described with respect to FIG. 5 above, such a setting may allow for slight overpressure in exchange for improved fluid flow rates.

And finally, FIG. 12D illustrates a “Low” pressure limit sensitivity setting. Under the “Low” setting, the system pressure is allowed to exceed the programmed pressure limit by a notable amount (e.g., about 15-20 psi), thereby prioritizing the fluid flow rates over the programmed pressure limit, similar to the example described above with respect to FIG. 6.

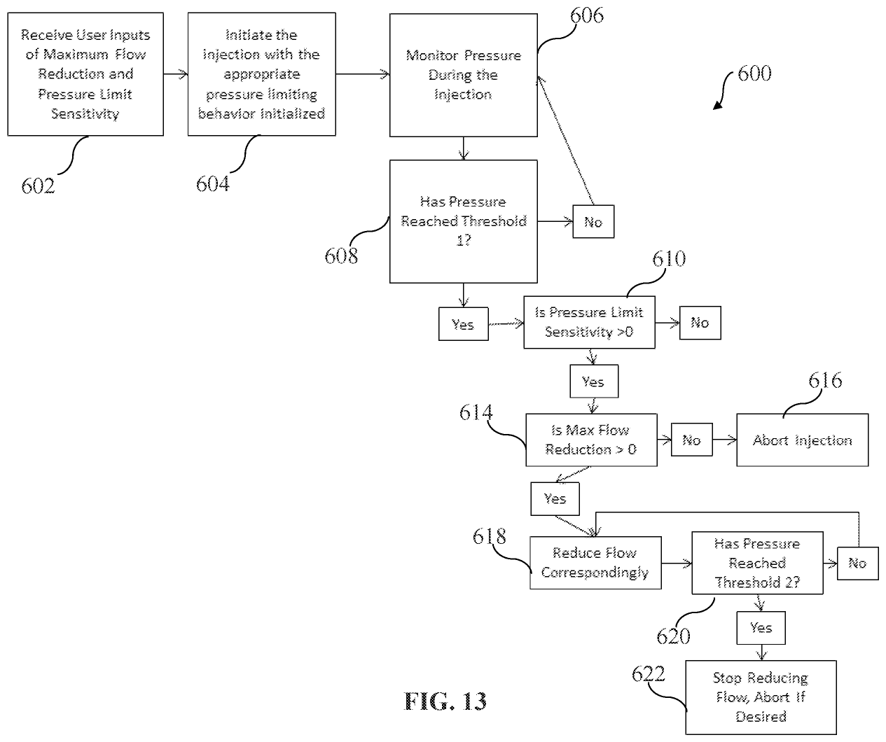

Next, referring to FIG. 13, an example logic workflow 600 in accordance with an embodiment of the disclosure is illustrated.

First, at 602, user inputs are received regarding the maximum flow reduction and pressure limit sensitivity. As described above, these user inputs may be received via, e.g., a GUI or other user interface. At 604, an injection procedure is initiated, with the appropriate pressure limiting behavior being initialized based on the user inputs.

At 606, fluid pressure during the injection procedure is monitored via any appropriate method. Then, at 608, a determination is made as to whether or not the determined pressure has reached a Threshold 1 pressure. In some embodiments, Threshold 1 is a predetermined pressure level below the programmed pressure limit. For example, Threshold 1 may be a pressure level that is, e.g., 1%-20% below the programmed pressure limit.

If no (i.e., Threshold 1 has not been reached), the workflow returns to 606 and the pressure continues to be monitored. However, if yes, a determination is made, at 610, whether or not the user- or system-input pressure limit sensitivity is greater than 0. In this particular example, a pressure limit sensitivity of 0 is considered to be the highest (or “High”) pressure sensitivity setting, wherein it is of the utmost importance during a particular injection procedure that the pressure limit may not be exceeded. If no (i.e., the pressure limit sensitivity is set to 0), the injection is aborted at 612. Alternatively, if yes (i.e., the pressure limit sensitivity is set to greater than 0), a determination is made, at 614, whether or not the maximum flow reduction is greater than 0. In this example, a maximum flow reduction of 0 is considered to be a user setting of no flow reduction allowed for the particular injection procedure. If no, (i.e., the maximum flow reduction is set to 0), the injection is aborted at 616. However, if yes (i.e., the maximum flow reduction is set to greater than 0), then the flow rate may be reduced at 618. Such a reduction in fluid flow rate is meant to correspondingly reduce the pressure during the injection such that the programmed pressure limit is not reached (or exceeded over a given threshold amount). As described above, the reduction in flow rate may be based on a predetermined flow reduction profile, which may be linear or non-linear (e.g., polynomial, exponential, logarithmic, etc.).

Next, at 620, a determination is made as to whether or not a Threshold 2 pressure has been reached. In some examples, the Threshold 2 pressure may be a pressure level that is, e.g., 1%-20% above the programmed pressure limit. However, it is to be understood that the Threshold 2 pressure may also be less than 1% above the programmed pressure limit, equal to the programmed pressure limit, or even below the programmed pressure limit, depending upon pressure limit sensitivity and the user settings. If yes (i.e., the Threshold 2 pressure has been reached), then, at 622, the system may stop the flow reduction and/or abort the injection procedure. However, if no (i.e., the Threshold 2 pressure has not been reached), then the workflow may return to 618, further reducing the fluid flow rate. The flow rate may continue to be reduced until either pressure Threshold 2 is reached, the maximum flow rate reduction is reached, or the determined pressure is below Threshold 1.

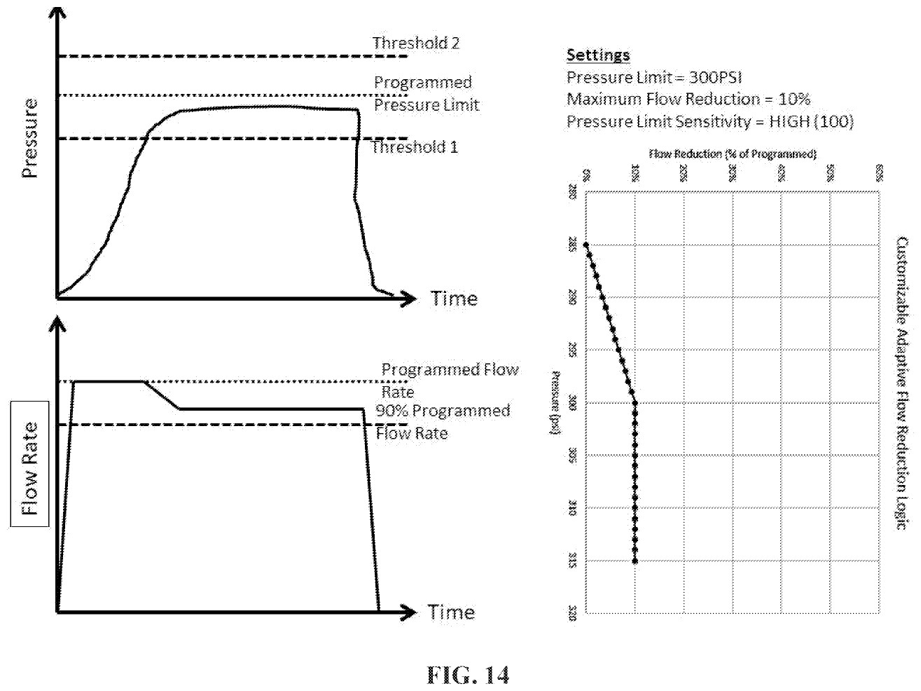

Referring now to FIGS. 14-20, example fluid injection scenarios in accordance with the embodiments described herein are illustrated.

First, referring to FIG. 14, settings of a maximum flow reduction of 10% and a “High” pressure limit sensitivity are input by the user as described above, with a programmed pressure limit of 300 PSI also being set. As shown in the corresponding pressure vs. time and flow rate vs. time graphs, during the injection procedure, as the flow rate rises to meet a programmed flow rate setting, the pressure also rises, and may eventually reach a Threshold 1 pressure below the programmed pressure limit. Once the Threshold 1 pressure is reached, the system is configured to reduce the flow rate in order to avoid reaching the programmed pressure limit. While the maximum flow reduction of 10% is set (i.e., 90% of the programmed flow rate), in the injection procedure shown in FIG. 14, the pressure stabilizes below the programmed pressure limit without a full 10% reduction in flow rate being needed. The pressure never reaches the programmed pressure limit therefore the maximum fluid flow reduction is never reached. Thus, the system is capable of maintaining a flow rate closer to the programmed flow rate, while still keeping the pressure below the programmed pressure limit.

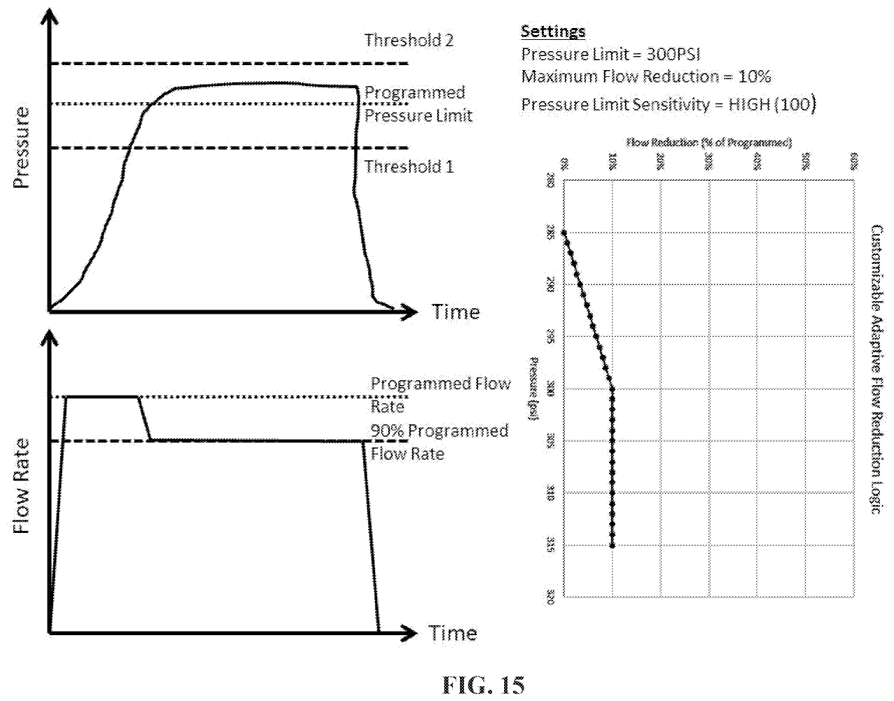

Referring to FIG. 15, an injection scenario similar to that described above with respect to FIG. 14 is illustrated, with settings of a maximum flow reduction of 10% and a “High” pressure limit sensitivity being input by the user, and a programmed pressure limit of 300 PSI also being set. However, unlike the injection scenario shown in FIG. 14, in FIG. 15, as the pressure reaches Threshold 1, the flow rate is reduced, but the programmed pressure limit is still reached. As the pressure limit sensitivity is set to “High”, all of the allowable flow rate reduction (10%) is achieved in the time interval between when the pressure reaches Threshold 1 and when the pressure reaches the programmed pressure limit. At that point, the flow rate is maintained at 90% of the programmed flow rate. However, as shown on the pressure vs. time graph, the pressure stabilizes at the 90% flow rate, with the pressure not reaching a Threshold 2 pressure. As the Threshold 2 pressure is not reached, the injection procedure is able to continue at the reduced flow rate without the need for the procedure to be aborted. According to this scenario, the programmed pressure limit is reached, however, since the pressure limit sensitivity is set to “High”, all of the allowable fluid flow rate reduction is achieved in the time interval between when the pressure reaches Threshold 1 and when the pressure reaches the programmed pressure limit.

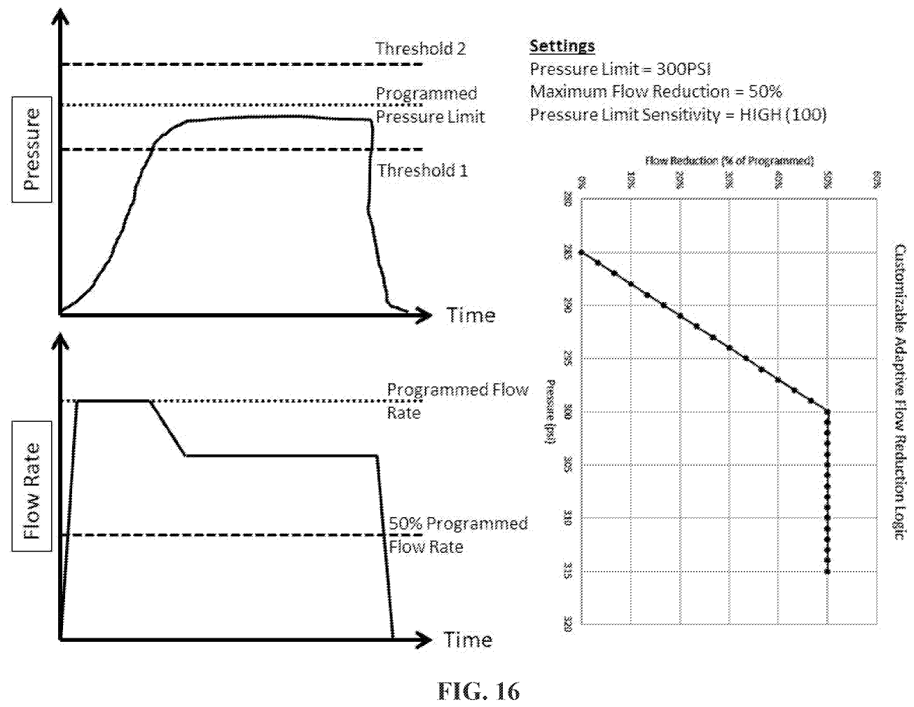

Next, referring to FIG. 16, an injection scenario is graphically illustrated in which a relatively high maximum flow reduction of 50% is set, along with a “High” pressure limit sensitivity. Similar to the scenario shown in FIG. 14 above, as the flow rate rises to meet a programmed flow rate setting, the pressure also rises, eventually reaching a Threshold 1 pressure below the programmed pressure limit. Once the Threshold 1 pressure is reached, the system is configured to reduce the flow rate in order to avoid reaching the programmed pressure limit. However, rather than abruptly reducing the flow rate to the maximum amount (50%), the system is configured for a more gradual (linear or non-linear) reduction in flow rate, thereby allowing the pressure to stabilize below the programmed pressure limit with far less than the 50% maximum reduction in flow rate being needed. According to this injection scenario, the pressure never reaches the programmed pressure limit and therefore the maximum fluid flow rate reduction is never reached. Thus, the system is capable of maintaining a flow rate closer to the programmed flow rate, while still keeping pressure below the programmed pressure limit.

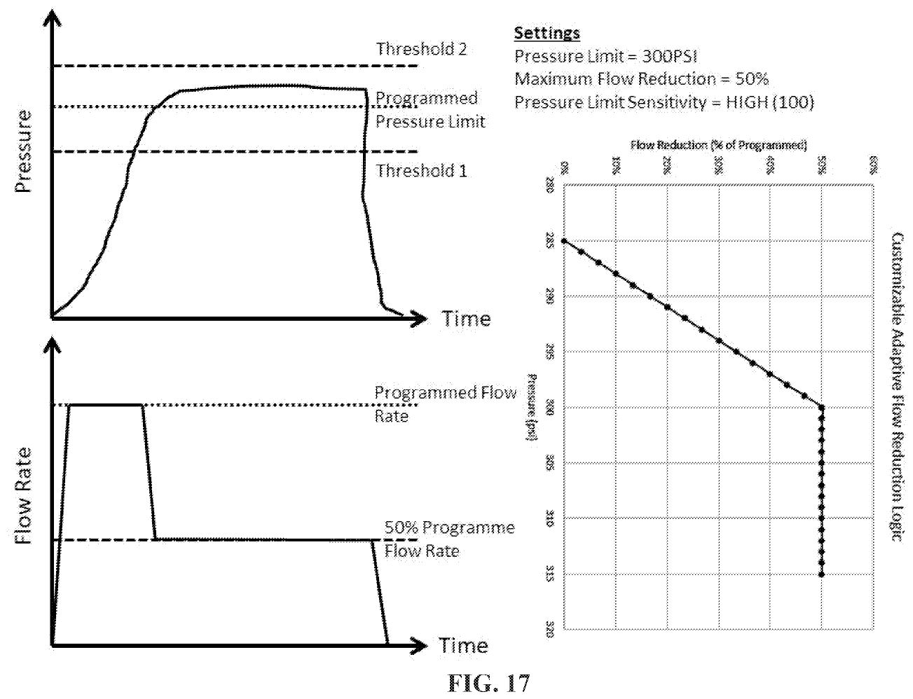

FIG. 17 shows an injection scenario similar to that described above with respect to FIG. 16, with a relatively high maximum flow reduction of 50% is set, along with a “High” pressure limit sensitivity. However, unlike the injection scenario shown in FIG. 16, in FIG. 17, as the pressure reaches Threshold 1, the flow rate is reduced, but the programmed pressure limit is still reached (and, in fact, is exceeded). As the pressure limit sensitivity is set to “High”, the full 50% of allowable flow rate reduction is achieved in the time interval between when the pressure reached Threshold 1 and when the pressure reached the programmed pressure limit. At that point, the flow rate is maintained at 50% of the programmed flow rate. However, as shown on the pressure vs. time graph, the pressure stabilizes at the 50% flow rate, not reaching a Threshold 2 pressure. As the Threshold 2 pressure is not reached, the injection procedure is able to continue at the reduced flow rate without the need for the procedure to be aborted. According to this injection scenario, the programmed pressure limit is reached, however, since the pressure limit sensitivity is set to “High”, all of the allowable fluid flow rate reduction is achieved in the time interval between when the pressure reaches Threshold 1 and when the pressure reaches the programmed pressure limit.

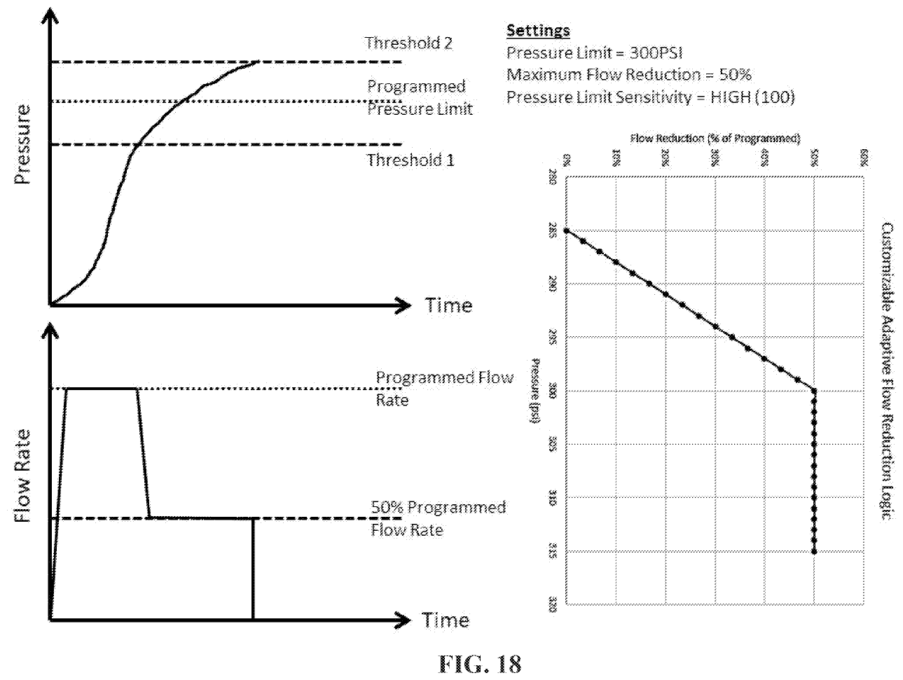

Conversely, referring to FIG. 18, an injection scenario having similar user settings is illustrated (i.e., maximum flow reduction=50%, pressure limit sensitivity=High), but even as the full 50% of allowable flow rate reduction is achieved in the time interval between when the pressure reaches Threshold 1 and when the pressure reached the programmed pressure limit, the pressure continues to increase. While the maximum 50% reduced flow rate is maintained, eventually the pressure increases until a Threshold 2 pressure is reached, at which point system is configured to abort the injection procedure, completely shutting of the fluid flow before the injection procedure is completed. According to this injection scenario, the programmed pressure limit is reached and since the pressure limit sensitivity is set to “High”, all of the allowable fluid flow rate reduction is achieved in the time interval between when the pressure reaches Threshold 1 and when the pressure reaches the programmed pressure limit, so when the pressure Threshold 2 is reached, the injection is aborted.

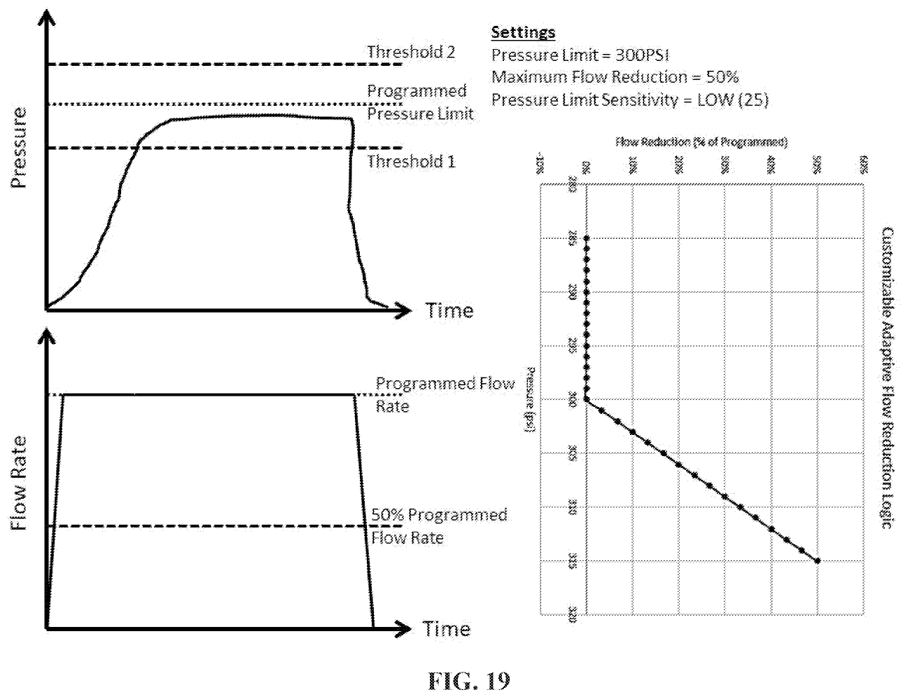

Next, referring to FIG. 19, an injection scenario in accordance with another aspect of the disclosure is illustrated. Unlike the injection scenarios described above with respect to FIGS. 14-18, the injection scenario of FIG. 19 pertains to an injection procedure in which the pressure limit sensitivity is set as “Low”, and the maximum flow reduction is 50% of the programmed flow rate. As is illustrated, as the flow rate increases over time to reach the programmed flow rate, the pressure also rises toward the programmed pressure limit, eventually crossing a Threshold 1 pressure. However, because the pressure limit sensitivity is “Low”, the system is configured to sustain the programmed flow rate, even after Threshold 1 pressure is reached. As is illustrated, the fluid pressure is eventually stabilized before the programmed pressure limit is reached. Thus, due to the “Low” pressure limit sensitivity, the injection procedure is able to be completed without a flow reduction being initiated. According to this injection scenario, the programmed pressure limit is never reached and therefore, the fluid flow reduction is never initiate because the pressure limit sensitivity is set to “Low”.

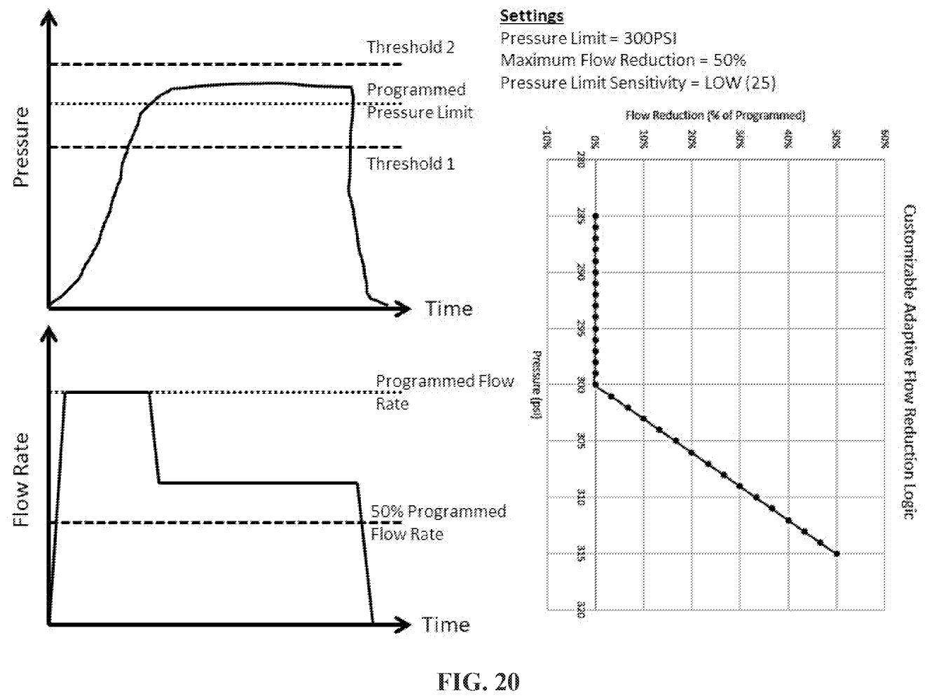

Finally, referring to FIG. 20, an injection scenario having similar user- or system-defined settings as those described in FIG. 19 is shown, with the pressure limit sensitivity being set as “Low”, and the maximum flow reduction being 50% of the programmed flow rate. However, in FIG. 20, the pressure eventually increases to a point at which the programmed pressure limit is reached. As the pressure limit sensitivity is set as “Low”, the flow reduction is not initiated until the programmed pressure limit is reached. As is shown, the pressure eventually stabilizes before reaching a Threshold 2 pressure, and without the maximum 50% flow reduction being needed to achieve such stabilization. Thus, as the pressure sensitivity is “Low”, the injection procedure is able to continue above the lowest possible flow rate and above the programmed pressure limit without the need for the procedure to be aborted. According to this injection scenario, the programmed pressure limit is reached and since the pressure limit sensitivity is set to “Low”, the fluid flow reduction is initiated when the pressure exceeds the programmed pressure limit. However, since the pressure Threshold 2 is never reached, the maximum fluid flow rate reduction is not achieved.

While FIGS. 14-20 describe injection scenarios relying on two threshold pressures (Threshold 1 and Threshold 2), it is to be understood that there may be more than two threshold pressures relative to the programmed pressure limit. For example, a third threshold pressure (Threshold 3) may be utilized such that any time Threshold 3 is reached, the injection procedure may be aborted. Depending upon the user- or system-defined settings, Threshold 3 may be different than Threshold 1, Threshold 2, and the programmed pressure limit, or it may be equal to any of Threshold 1, Threshold 2, or the programmed pressure limit. According to certain embodiments, there may be a third pressure threshold (Threshold 3) at a pressure value between the programmed pressure limit and Threshold 2; at a pressure between the programmed pressure limit and Threshold 1; or may be greater than Threshold 2.

Although the disclosure has been described in detail for the purpose of illustration based on what is currently considered to be the most practical and preferred embodiments or aspects, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

1. A fluid injector system configured for administering at least one fluid, the fluid injector system comprising:
at least one fluid injector device;
at least one user interface; and
a control device in communication with the at least one user interface, the control device comprising at least one processor programmed or configured to utilize at least one of internal inputs and external inputs to instruct the at least one fluid injector device to perform a fluid injection procedure according to a profile that determines prioritization of maintaining flow rate or limiting a fluid delivery pressure.

2. The fluid injector system of claim 1, wherein the at least one of internal inputs and external inputs are selected from the group consisting of receiving a maximum pressure limit for a fluid injection procedure; receiving a programmed fluid flow rate for the fluid injection procedure; receiving a maximum fluid flow rate reduction input for the fluid injection procedure, wherein the maximum fluid flow rate reduction input is selected by a user via the at least one user interface or by the system based off of at least one of patient information and system injection parameters; receiving a pressure limit sensitivity input for the fluid injection procedure, wherein the pressure limit sensitivity input is selected by the user via the at least one user interface; and any combination thereof,
wherein the control device is further configured to instruct the at least one fluid injector device to perform the injection procedure based on one or more of the maximum pressure limit, the programmed fluid flow rate, the maximum fluid flow rate reduction input, and the pressure limit sensitivity input.

3. The fluid injector system of claim 1, wherein the at least one user interface comprises at least one graphical user interface and wherein the at least one graphical user interface displays a flow rate profile of the fluid injection procedure,
wherein the displayed flow rate profile further indicates one or more of the maximum pressure limit, the programmed fluid flow rate, the maximum fluid flow rate reduction input, and the pressure limit sensitivity input.

4. The fluid injector system of claim 1, wherein the at least one processor is programmed or configured to limiting a fluid delivery pressure by reducing a flow rate of the fluid, diluting a more viscous fluid with a less viscous fluid, or combinations thereof.

5. The fluid injector system of claim 1, wherein the at least one processor is further programmed or configured to reduce the fluid flow rate of the at least one fluid injector device to provide a reduction in flow rate below the programmed fluid flow rate when a predetermined threshold pressure relative to the maximum pressure limit is reached.

6. The fluid injector system of claim 5, wherein the reduction in fluid flow rate has one of a linear or a non-linear flow reduction profile.

7. The fluid injector system of claim 5, wherein the reduction of fluid flow rate is based on at least one input provided by a user of the fluid injector device.

8. (canceled)

9. The fluid injector system of claim 7, wherein the at least one user input provided by the user is selected by the user from a series of inputs saved in a protocol library or inputted manually by the user prior to initiation of the fluid injection procedure.

10. (canceled)

11. The fluid injector system of claim 5, wherein the reduction in fluid flow rate is based on at least one internally sourced or at least one externally sourced input, and wherein the at least one internally sourced or the at least one externally sourced input is selected from the group consisting of historical injector data, historical patient data, information from one or more barcodes, one or more lookup tables, and combinations of any thereof,
wherein the one or more barcodes is located on a patient record, the fluid injector, a medical fluid container, a sterile disposable, [[and]] or combinations thereof.

12-14. (canceled)

15. The fluid injector system of claim 5, wherein the at least one processor is programmed or configured to abort the injection procedure if specific configuration criteria are met and the pressure is detected to equal or exceed the predetermined pressure threshold.

16. A method of user- or system-configurable pressure limiting behavior for a fluid injector system configured for administering at least one fluid comprising:
providing at least one of internal inputs and external inputs to a fluid injector system;
prioritizing maintaining flow rate or limiting a fluid delivery pressure based on the at least one of the internal inputs and the external inputs;
developing an injection profile based on the prioritizing; and
instructing an at least one fluid injector device to perform a fluid injection procedure according to the injection profile.

17. The method of claim 16, wherein providing the at least one of internal inputs and external inputs comprises an operation selected from the group consisting of:
inputting or selecting a maximum pressure limit for an injection procedure into at least one user interface in communication with a control device of the fluid injector system, wherein the control device comprises at least one processor;
inputting or selecting a programmed fluid flow rate for injecting at least one fluid from the at least one fluid injector device;
inputting or selecting a maximum fluid flow rate reduction input for the injection procedure based off of at least one of patient information and system injection parameters;
inputting or selecting a pressure limit sensitivity input for the injection procedure; and
combinations of any thereof.

18. The method of claim 17 further comprising generating, by the control device, instructions to the fluid injector system to perform the injection procedure based on one or more of the maximum pressure limit, the programmed fluid flow rate, the maximum fluid flow rate reduction input, and the pressure limit sensitivity input.

19. The method of claim 16, wherein the at least on user interface comprises at least one graphical user interface and wherein the at least one graphical user interface displays a flow rate profile of the fluid injection procedure,
wherein the displayed flow rate profile further indicates one or more of the maximum pressure limit, the programmed fluid flow rate, the maximum fluid flow rate reduction input, and the pressure limit sensitivity input.

20. The method of claim 16, further comprising limiting a fluid delivery pressure by reducing a flow rate of the fluid, diluting a more viscous fluid with a less viscous fluid, or combinations thereof.

21. The method of claim 16, further comprising reducing, by the control device, the fluid flow rate of the at least one fluid injector device to provide a reduction in flow rate below the programmed fluid flow rate when a predetermined threshold pressure relative to the maximum pressure limit is reached.

22. The method of claim 21, wherein reducing the fluid flow rate is based on at least one input provided by a user of the fluid injector device.

23. (canceled)

24. The method of claim 22, wherein the at least one user input provided by the user is selected by the user from a series of inputs saved in a protocol library or inputted manually by the user prior to initiation of the fluid injection procedure.

25. (canceled)

26. The method of claim 21, wherein reducing the fluid flow rate is based on at least one internally sourced or at least one externally sourced input, and
wherein the at least one internally sourced or the at least one externally sourced input is selected from the group consisting of historical injector data, historical patient data, information from one or more barcodes, one or more lookup tables, and combinations of any thereof,
wherein the one or more barcodes is located on a patient record, the fluid injector, a medical fluid container, a sterile disposable, and combinations thereof.

27-29. (canceled)

30. The method of claim 21, further comprising aborting the injection procedure if specific configuration criteria are met and the pressure is detected to exceed the predetermined pressure threshold.
